SEQUENCE LISTING (11 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 445 aa, Artificial
  anti-PD-1 antibody sequence
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 218 aa, Artificial
  anti-PD-1 antibody sequence
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 119 aa, Artificial
  anti-PD-1 antibody sequence
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 5 - protein, 111 aa, Artificial
  anti-PD-1 antibody sequence
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 6 - protein, 5 aa, Artificial
  anti-PD-1 antibody sequence
SYWMN

SEQ ID NO: 7 - protein, 17 aa, Artificial
  anti-PD-1 antibody sequence
VIHPSDSETWLDQKFKD

SEQ ID NO: 8 - protein, 10 aa, Artificial
  anti-PD-1 antibody sequence
EHYGTSPFAY

SEQ ID NO: 9 - protein, 16 aa, Artificial
  anti-PD-1 antibody sequence
RASESVDNYGMSFMNW

SEQ ID NO: 10 - protein, 7 aa, Artificial
  anti-PD-1 antibody sequence
AASNQGS

SEQ ID NO: 11 - protein, 9 aa, Artificial
  anti-PD-1 antibody sequence
QQSKEVPYT

(19) United States
(12) Patent Application Publication
(10) Pub. No.: US 2021/0030869 A1
(43) Pub. Date: Feb. 4, 2021

(54) DOSING REGIMEN FOR AN IDO INHIBITOR

(71) Applicant: Incyte Corporation, Wilmington, DE (US)

(72) Inventors: Michael Smith, Pottstown, PA (US); Robert C. Newton, Avondale, PA (US); Sherry Owens, Wilmington, DE (US)

(21) Appl. No.: 16/944,953

(22) Filed: Jul. 31, 2020

Related U.S. Application Data
(60) Provisional application No. 62/881,518, filed on Aug. 1, 2019.

(51) Int. Cl. A61K 39/395; A61K 31/4245; A61K 9/00; A61P 35/00

(52) U.S. Cl. CPC A61K 39/3955; A61P 35/00; A61K 9/0019; A61K 31/4245

(57) ABSTRACT

The present disclosure relates to dosing regimens for treating cancer by administering epacadostat in combination with an antibody, or an antibody fragment thereof, that binds to PD-1.

### CROSS-REFERENCE TO RELATED APPLICATIONS

This application claims the benefit of U.S. Provisional Application Ser. No. 62/881,518, filed Aug. 1, 2019, the disclosure of which is incorporated herein by reference in its entirety.

### FIELD OF INVENTION

The present disclosure relates to dosing regimens for treating cancer by administering epacadostat in combination with an antibody, or an antibody fragment thereof, that binds to PD-1.

### BACKGROUND OF THE INVENTION

Tryptophan (Trp) is an essential amino acid required for the biosynthesis of proteins, niacin and the neurotransmitter 5-hydroxytryptamine (serotonin). The enzyme indoleamine 2,3-dioxygenase (also known as INDO, IDO or IDO1) catalyzes the first and rate limiting step in the degradation of L-tryptophan to N-formyl-kynurenine. In human cells, a depletion of Trp resulting from IDO activity is a prominent gamma interferon (IFN-γ)—inducible antimicrobial effector mechanism. IFN-γ stimulation induces activation of IDO, which leads to a depletion of Trp, thereby arresting the growth of Trp-dependent intracellular pathogens such as Toxoplasma gondii and Chlamydia trachomatis. IDO activity also has an antiproliferative effect on many tumor cells, and IDO induction has been observed in vivo during rejection of allogeneic tumors, indicating a possible role for this enzyme in the tumor rejection process (Daubener, et al., 1999, Adv. Exp. Med. Biol., 467: 517-24; Taylor, et al., 1991, FASEB J., 5: 2516-22).

It has been observed that HeLa cells co-cultured with peripheral blood lymphocytes (PBLs) acquire an immuno-inhibitory phenotype through up-regulation of IDO activity. A reduction in PBL proliferation upon treatment with interleukin-2 (IL2) was believed to result from IDO released by the tumor cells in response to IFNG secretion by the PBLs. This effect was reversed by treatment with 1-methyl-tryptophan (1MT), a specific IDO inhibitor. It was proposed that IDO activity in tumor cells may serve to impair antitumor responses (Logan, et al., 2002, Immunology, 105: 478-87).

Recently, an immunoregulatory role of Trp depletion has received much attention. Several lines of evidence suggest that IDO is involved in induction of immune tolerance. Studies of mammalian pregnancy, tumor resistance, chronic infections and autoimmune diseases have shown that cells expressing IDO can suppress T-cell responses and promote tolerance. For example, increased levels of IFNs and elevated levels of urinary Trp metabolites have been observed in autoimmune diseases; it has been postulated that systemic or local depletion of Trp occurring in autoimmune diseases may relate to the degeneration and wasting symptoms of these diseases.

Further evidence for a tumoral immune resistance mechanism based on tryptophan degradation by IDO comes from the observation that most human tumors constitutively express IDO, and that expression of IDO by immunogenic mouse tumor cells prevents their rejection by preimmunized mice. This effect is accompanied by a lack of accumulation of specific T cells at the tumor site and can be partly reverted by systemic treatment of mice with an inhibitor of IDO, in the absence of noticeable toxicity. Thus, it was suggested that the efficacy of therapeutic vaccination of cancer patients might be improved by concomitant administration of an IDO inhibitor (Uyttenhove et al., 2003, Nature Med., 9: 1269-74). It has also been shown that the IDO inhibitor, 1-MT, can synergize with chemotherapeutic agents to reduce tumor growth in mice, suggesting that IDO inhibition may also enhance the anti-tumor activity of conventional cytotoxic therapies (Muller et al., 2005, Nature Med., 11: 312-9).

One mechanism contributing to immunologic unresponsiveness toward tumors may be presentation of tumor antigens by tolerogenic host APCs. A subset of human IDO-expressing antigen-presenting cells (APCs) that coexpressed CD123 (IL3RA) and CCR6 and inhibited T-cell proliferation have also been described. Both mature and immature CD123-positive dendritic cells suppressed T-cell activity, and this IDO suppressive activity was blocked by 1MT (Munn, et al., 2002, Science, 297: 1867-70). It has also been demonstrated that mouse tumor-draining lymph nodes (TDLNs) contain a subset of plasmacytoid dendritic cells (pDCs) that constitutively express immunosuppressive levels of IDO. Despite comprising only 0.5% of lymph node cells, in vitro, these pDCs potently suppressed T cell responses to antigens presented by the pDCs themselves and also, in a dominant fashion, suppressed T cell responses to third-party antigens presented by nonsuppressive APCs. Within the population of pDCs, the majority of the functional IDO-mediated suppressor activity segregated with a novel subset of pDCs coexpressing the B-lineage marker CD19. Thus, it was hypothesized that IDO-mediated suppression by pDCs in TDLNs creates a local microenvironment that is potently suppressive of host antitumor T cell responses (Munn, et al., 2004, J. Clin. Invest., 114(2): 280-90).

IDO degrades the indole moiety of tryptophan, serotonin and melatonin, and initiates the production of neuroactive and immunoregulatory metabolites, collectively known as kynurenines. By locally depleting tryptophan and increasing proapoptotic kynurenines, IDO expressed by dendritic cells (DCs) can greatly affect T-cell proliferation and survival. IDO induction in DCs could be a common mechanism of deletional tolerance driven by regulatory T cells. Because such tolerogenic responses can be expected to operate in a variety of physiopathological conditions, tryptophan metabolism and kynurenine production might represent a crucial interface between the immune and nervous systems (Grohmann, et al., 2003, Trends Immunol., 24: 242-8). In states of persistent immune activation, availability of free serum Trp is diminished and, as a consequence of reduced serotonin production, serotonergic functions may also be affected (Wirleitner, et al., 2003, Curr. Med. Chem., 10: 1581-91).

In light of the experimental data indicating a role for IDO in immunosuppression and tumor resistance and/or rejection, therapeutic agents aimed at suppression of tryptophan degradation by inhibiting IDO activity are desirable. One potent inhibitor of IDO1 is epacadostat (INCB24360; 4-({2-[(aminosulfonyl)amino]ethyl}amino)-N-(3-bromo-4-fluorophenyl)-N′-hydroxy-1,2,5-oxadiazole-3-carboximidamide), which has the formula below:

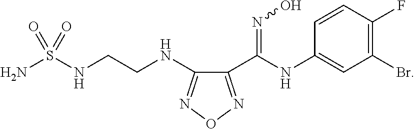 [SMILES: NS(=O)(=O)NCCNc1nonc1C(=NO)Nc1ccc(F)c(Br)c1]

There remains a need for new treatment regimens for cancer using IDO1 inhibitors. The present disclosure is directed toward this need and others.

### SUMMARY

The present disclosure provides, inter alia, methods of treating cancer in a patient comprising administering to said patient:
(i) epacadostat, or a pharmaceutically acceptable salt thereof, at a dose from about 400 mg to about 700 mg on a free base basis BID; and
(ii) an antibody that binds to human PD-1, wherein the antibody comprises (ii-1) a variable heavy (VH) domain comprising VH complementarity determining region (CDR)1, VH CDR2, and VH CDR3; and (ii-2) a variable light (VL) domain comprising VL CDR1, VL CDR2, and VL CDR3; wherein:
   (a) the VH CDR1 comprises the amino acid sequence SYWMN (SEQ ID NO:6);
   (b) the VH CDR2 comprises the amino acid sequence VIHPSDSETWLDQKFKD (SEQ ID NO:7);
   (c) the VH CDR3 comprises the amino acid sequence EHYGTSPFAY (SEQ ID NO:8);
   (d) the VL CDR1 comprises the amino acid sequence RASESVDNYGMSFMNW (SEQ ID NO:9);
   (e) the VL CDR2 comprises the amino acid sequence AASNQGS (SEQ ID NO:10); and
   (f) the VL CDR3 comprises the amino acid sequence QQSKEVPYT (SEQ ID NO:11).

In some embodiments, the epacadostat, or a pharmaceutically acceptable salt thereof, is administered at a dose from about 600 mg on a free base basis BID.

### DETAILED DESCRIPTION

The present disclosure further provides a method of treating cancer in a patient comprising administering to said patient:

(i) epacadostat, or a pharmaceutically acceptable salt thereof, at a dose from about 400 mg to about 700 mg on a free base basis BID; and

(ii) an antibody that binds to human PD-1, which is ANTIBODY X. ANTIBODY X is retifanlimab. Unexpectedly, doses of epacadostat in the methods of present disclosure (e.g., 600 mg) have been shown to unexpectedly lower the kynurenine levels relative to lower doses (e.g., 100 mg BID) (see Example 1 infra) when administered in combination with ANTIBODY X. While not wanting to be bound by any particular theory, the claimed doses of epacadostat are thought to work by blocking the additional IDO1 activity induced as a result of an immune system stimulant such as ANTIBODY X.

The amino acid sequence of the human PD-1 protein (Genbank Accession No. NP_005009) is:

(SEQ ID NO: 1)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTARPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

ANTIBODY X is a humanized, IgG4 monoclonal antibody that binds to human PD-1 (see WO2017019846, which is incorporated herein by reference in its entirety). The amino acid sequences of the mature ANTIBODY X heavy and light chains is described below.

Complementarity-determining regions (CDRs) 1, 2, and 3 of the variable heavy (VH) domain and the variable light (VL) domain are shown in that order from N to the C-terminus of the mature VL and VH sequences and are both underlined and boldened. An antibody consisting of the mature heavy chain (SEQ ID NO:2) and the mature light chain (SEQ ID NO:3) listed below is termed ANTIBODY X.

Mature ANTIBODY X heavy chain (HC)
(SEQ ID NO: 2)
QVQLVQSGAEVKKPGASVKVSCKASGYSFTSYWMNWVRQAPGQGLEWIGV
IHPSDSETWLDQKFKDRVTITVDKSTSTAYMELSSLRSEDTAVYYCAREH
YGTSPFAYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLEPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFELYSRLTVDKSRWQEGNVFSCSVMHEALHNHYTQKSLSLSLG
Mature ANTIBODY X light chain (LC)
(SEQ ID NO: 3)
EIVLTQSPATLSLSPGERATLSCRASESVDNYGMSFMNWFQQKPGQPPKL
LIHAASNQGSGVPSRFSGSGSGTDFTLTISSLEPEDFAVYFCQQSKEVPY
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

The variable heavy (VH) domain of ANTIBODY X has the following amino acid sequence:

(SEQ ID NO: 4)
QVQLVQSGAEVKKPGASVKVSCKASGYSFTSYWMNWVRQAPGQGLEWIGV
IHPSDSETWLDQKFKDRVTITVDKSTSTAYMELSSLRSEDTAVYYCAREH
YGTSPFAYWGQGTLVTVSS

The variable light (VL) domain of ANTIBODY X has the following amino acid sequence:

(SEQ ID NO: 5)
EIVLTQSPATLSLSPGERATLSCRASESVDNYGMSFMNWFQQKPGQPPKL
LIHAASNQGSGVPSRFSGSGSGTDFTLTISSLEPEDFAVYFCQQSKEVPY
TFGGGTKVEIK

The amino acid sequences of the VH CDRs of ANTIBODY X are listed below:

VH CDR1:
(SEQ ID NO: 6)
SYWMN;
VH CDR2:
(SEQ ID NO: 7)
VIHPSDSETWLDQKFKD;
VH CDR3:
(SEQ ID NO: 8)
EHYGTSPFAY

The amino acid sequences of VL CDRs of ANTIBODY X are listed below:

VL CDR1:
(SEQ ID NO: 9)
RASESVDNYGMSFMNW;
VL CDR2:
(SEQ ID NO: 10)
AASNQGS;
and
VL CDR3:
(SEQ ID NO: 11)
QQSKEVPYT.

Accordingly, the present disclosure provides a method of treating cancer in a patient, comprising administering to said patient:

(i) epacadostat, or a pharmaceutically acceptable salt thereof, at a dose from about 400 mg to about 700 mg on a free base basis BID; and

(ii) an antibody that binds to human PD-1, wherein the antibody comprises (ii-1) a variable heavy (VH) domain comprising VH complementarity determining region (CDR)1, VH CDR2, and VH CDR3; and (ii-2) a variable light (VL) domain comprising VL CDR1, VL CDR2, and VL CDR3; wherein:

(a) the VH CDR1 comprises the amino acid sequence SYWMN (SEQ ID NO:6);

(b) the VH CDR2 comprises the amino acid sequence VIHPSDSETWLDQKFKD (SEQ ID NO:7);

(c) the VH CDR3 comprises the amino acid sequence EHYGTSPFAY (SEQ ID NO:8);

(d) the VL CDR1 comprises the amino acid sequence RASESVDNYGMSFMNW (SEQ ID NO:9);

(e) the VL CDR2 comprises the amino acid sequence AASNQGS (SEQ ID NO:10); and

(f) the VL CDR3 comprises the amino acid sequence QQSKEVPYT (SEQ ID NO:11).

In some embodiments, the antibody comprises an Fc Region wherein the Fc Region is of the IgG4 isotype. In some embodiments, the antibody comprises an Fc Region of the IgG4 isotype and an IgG4 Hinge Domain that comprises a stabilizing mutation. In some embodiments, the antibody comprises an Fc Region of the IgG4 isotype and an IgG4 Hinge Domain that comprises a S228P substitution (see, e.g., SEQ ID NO:13: ESKYGPPCPPCP, (Lu et al, (2008) “The Effect Of A Point Mutation On The Stability Of IgG4 As Monitored By Analytical Ultracentrifugation,” J. Pharmaceutical Sciences 97:960-969) to reduce the incidence of strand exchange.

In some embodiments, the epacadostat, or a pharmaceutically acceptable salt thereof, and the ANTIBODY X are administered to a patient simultaneously or sequentially. In some embodiments, the epacadostat, or a pharmaceutically acceptable salt thereof, and the ANTIBODY X are administered to a patient simultaneously. In some embodiments, the epacadostat, or a pharmaceutically acceptable salt thereof, and the ANTIBODY X are administered to a patient sequentially.

In some embodiments, the cancer is a solid tumor.

In some embodiments, the VH domain comprises the amino acid sequence set forth in SEQ ID NO:4.

In some embodiments, the antibody comprises a heavy chain, wherein the heavy chain comprises the amino acid sequence set forth in SEQ ID NO:2.

In some embodiments, the VL domain comprises the amino acid sequence set forth in SEQ ID NO:5.

In some embodiments, the antibody comprises a light chain, wherein the light chain comprises the amino acid sequence set forth in SEQ ID NO:3.

In some embodiments, the VH domain comprises the amino acid sequence set forth in SEQ ID NO:4 and the VL domain comprises the amino acid sequence set forth in SEQ ID NO:5.

In some embodiments, the antibody comprises a heavy chain and a light chain, and wherein the heavy chain comprises the amino acid sequence set forth in SEQ ID NO:2 and the light chain comprises the amino acid sequence set forth in SEQ ID NO:3.

In some embodiments, antibody is a humanized antibody.

In some embodiments, the epacadostat, or a pharmaceutically acceptable salt thereof, is administered at a dose of about 500 mg to about 700 mg on a free base basis BID.

In some embodiments, the epacadostat, or a pharmaceutically acceptable salt thereof, is administered at a dose of about 400 mg to about 600 mg on a free base basis BID.

In some embodiments, the epacadostat, or a pharmaceutically acceptable salt thereof, is administered at a dose of about 500 mg to about 600 mg on a free base basis BID.

In some embodiments, the epacadostat, or a pharmaceutically acceptable salt thereof, is administered at a dose of about 400 mg to about 600 mg on a free base basis BID.

In some embodiments, the epacadostat, or a pharmaceutically acceptable salt thereof, is administered at a dose of about 550 mg to about 650 mg on a free base basis BID.

In some embodiments, the epacadostat, or a pharmaceutically acceptable salt thereof, is administered at a dose of about 575 mg to about 625 mg on a free base basis BID.

In some embodiments, the epacadostat, or a pharmaceutically acceptable salt thereof, is administered at a dose of about 400 mg on a free base basis BID.

In some embodiments, the epacadostat, or a pharmaceutically acceptable salt thereof, is administered at a dose of about 425 mg on a free base basis BID.

In some embodiments, the epacadostat, or a pharmaceutically acceptable salt thereof, is administered at a dose of about 450 mg on a free base basis BID.

In some embodiments, the epacadostat, or a pharmaceutically acceptable salt thereof, is administered at a dose of about 475 mg on a free base basis BID.

In some embodiments, the epacadostat, or a pharmaceutically acceptable salt thereof, is administered at a dose of about 500 mg on a free base basis BID.

In some embodiments, the epacadostat, or a pharmaceutically acceptable salt thereof, is administered at a dose of about 525 mg on a free base basis BID.

In some embodiments, the epacadostat, or a pharmaceutically acceptable salt thereof, is administered at a dose of about 550 mg on a free base basis BID.

In some embodiments, the epacadostat, or a pharmaceutically acceptable salt thereof, is administered at a dose of about 575 mg on a free base basis BID.

In some embodiments, the epacadostat, or a pharmaceutically acceptable salt thereof, is administered at a dose of about 600 mg on a free base basis BID.

In some embodiments, the epacadostat, or a pharmaceutically acceptable salt thereof, is administered at a dose of about 625 mg on a free base basis BID.

In some embodiments, the epacadostat, or a pharmaceutically acceptable salt thereof, is administered at a dose of about 650 mg on a free base basis BID.

In some embodiments, the epacadostat, or a pharmaceutically acceptable salt thereof, is administered at a dose of about 675 mg on a free base basis BID.

In some embodiments, the epacadostat, or a pharmaceutically acceptable salt thereof, is administered at a dose of about 700 mg on a free base basis BID.

In some embodiments, the epacadostat is administered as the free base.

In some embodiments, the epacadostat is administered at a dose of about 400 mg BID.

In some embodiments, the epacadostat is administered at a dose of about 425 mg BID.

In some embodiments, the epacadostat is administered at a dose of about 450 mg BID.

In some embodiments, the epacadostat is administered at a dose of about 475 mg BID.

In some embodiments, the epacadostat is administered at a dose of about 500 mg BID.

In some embodiments, the epacadostat is administered at a dose of about 525 mg BID.

In some embodiments, the epacadostat is administered at a dose of about 550 mg BID.

In some embodiments, the epacadostat is administered at a dose of about 575 mg BID.

In some embodiments, the epacadostat is administered at a dose of about 600 mg BID.

In some embodiments, the epacadostat is administered at a dose of about 625 mg BID.

In some embodiments, the epacadostat is administered at a dose of about 650 mg BID.

In some embodiments, the epacadostat is administered at a dose of about 675 mg BID.

In some embodiments, the epacadostat is administered at a dose of about 700 mg BID.

In some embodiments, the epacadostat, or a pharmaceutically acceptable salt thereof, is administered as a pharmaceutical composition. In some embodiments, the epacadostat, or a pharmaceutically acceptable salt thereof, is administered orally. In some embodiments, the epacadostat, or a pharmaceutically acceptable salt thereof, is administered as a solid oral dosage form. In some embodiments, the solid oral dosage form is a tablet or a capsule. In some embodiments, the solid oral dosage form is a tablet. In some embodiments, multiple tablets are administered to achieve a desired dose.

The anti-PD-1 antibody or antigen-binding fragment thereof can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody or antigen-binding fragment thereof can also be tailored for the individual case, e.g., by monitoring the subject, e.g., using tomographic imaging, e.g., to visualize a tumor.

The antibody or antigen-binding fragment can be administered as a fixed dose, or in a mg/kg patient weight dose. The dose can also be chosen to reduce or avoid production of antibodies against the antibody or antigen-binding fragment. Dosage regimens are adjusted to provide the desired response, e.g., a therapeutic response or a combinatorial therapeutic effect. Generally, doses of the antibody or antigen-binding fragment (and optionally a second agent) can be used in order to provide a subject with the agent in bioavailable quantities. For example, doses in the range of about 0.1-100 mg/kg, about 0.5-100 mg/kg, about 1 mg/kg-100 mg/kg, about 0.5-20 mg/kg, about 0.1-10 mg/kg, or about 1-10 mg/kg can be administered. Other doses can also be used. In specific embodiments, a subject in need of treatment is administered the antibody or antigen-binding fragment at a dose of about 1 mg/kg, about 2 mg/kg, about 3 mg/kg, about 4 mg/kg, about 5 mg/kg, about 10 mg/kg, about 15 mg/kg, about 20 mg/kg, about 30 mg/kg, about 35 mg/kg, or about 40 mg/kg. With respect to doses or dosages, the term “about” is intended to denote a range that is ±10% of a recited dose, such that, for example, a dose of about 3 mg/kg will be between 2.7 mg/kg and 3.3 mg/kg patient weight.

A composition may comprise about 1 mg/mL to 100 mg/ml or about 10 mg/mL to 100 mg/ml or about 50 to 250 mg/mL or about 100 to 150 mg/ml or about 100 to 250 mg/ml of antibody or antigen-binding fragment.

Dosage unit form or “fixed dose” or “flat dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody or antigen-binding fragment thereof may be administered via continuous infusion. Exemplary fixed doses include about 375 mg, about 500 mg and about 750 mg. With respect to doses or dosages, the term “about” is intended to denote a range that is ±10% of a recited dose, such that, for example, a dose of about 375 mg will be between 337.5 mg and 412.5 mg.

The antibody or antigen-binding fragment dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

In some embodiments of any of the above aspects, the antibody or antigen-binding fragment is administered at a fixed dose of about 375 mg once every 3 weeks.

In some embodiments of any of the above aspects, the antibody or antigen-binding fragment is administered at a fixed dose of about 500 mg once every 4 weeks.

In some embodiments of any of the above aspects, the antibody or antigen-binding fragment is administered at a fixed dose of about 750 mg once every 4 weeks.

In some embodiments of any of the above aspects, the antibody or antigen-binding fragment is administered at a dose of about 1 mg/kg once every 2 weeks.

In some embodiments of any of the above aspects, the antibody or antigen-binding fragment is administered at a dose of about 3 mg/kg once every 2 weeks.

In some embodiments of any of the above aspects, the antibody or antigen-binding fragment is administered at a dose of about 3 mg/kg once every 4 weeks.

In some embodiments of any of the above aspects, the antibody or antigen-binding fragment is administered at a dose of about 10 mg/kg once every 2 weeks.

In some embodiments of any of the above aspects, the antibody or antigen-binding fragment is administered at a dose of about 10 mg/kg once every 4 weeks.

In some embodiments of any of the above aspects, the antibody or antigen-binding fragment is administered at a fixed dose of about 375 mg once every 3 weeks.

In some embodiments of any of the above aspects, the antibody or antigen-binding fragment is administered at a fixed dose of about 500 mg once every 4 weeks.

In some embodiments of any of the above aspects, the antibody or antigen-binding fragment is administered at a fixed dose of about 750 mg once every 4 weeks.

In some embodiments, the term “about” refers to plus or minus 10% of the value. A skilled person in the art would know that the values presented herein can vary due to the conditions of the experiments such as variability in data collection or instruments.

### Epacadostat

Epacadostat can be synthesized as described in U.S. Pat. Nos. 8,088,803 and 9,321,755, which are incorporated herein by reference in their entirety.

The present disclosure also includes pharmaceutically acceptable salts of epacadostat described herein.

In some embodiments, epacadostat and salts thereof are substantially isolated. By “substantially isolated” is meant that the compound is at least partially or substantially separated from the environment in which it was formed or detected. Partial separation can include, for example, a composition enriched in epacadostat. Substantial separation can include compositions containing at least about 50%, at least about 60%, at least about 70%, at least about 80%, at least about 90%, at least about 95%, at least about 97%, or at least about 99% by weight of epacadostat, or salt thereof. Methods for isolating compounds and their salts are routine in the art.

Epacadostat can exist in various solid forms. As used herein “solid form” is meant to refer to a solid characterized by one or more properties such as, for example, melting point, solubility, stability, crystallinity, hygroscopicity, water content, TGA features, DSC features, DVS features, XRPD features, etc. Solid forms, for example, can be amorphous, crystalline, or mixtures thereof.

Different crystalline solid forms typically have different crystalline lattices (e.g., unit cells) and, usually as a result, have different physical properties. In some instances, different crystalline solid forms have different water or solvent content. The different crystalline lattices can be identified by solid state characterization methods such as by X-ray powder diffraction (XRPD). Other characterization methods such as differential scanning calorimetry (DSC), thermogravimetric analysis (TGA), dynamic vapor sorption (DVS), and the like further help identify the solid form as well as help determine stability and solvent/water content.

In some embodiments, the solid form is a crystalline solid. In some embodiments, epacadostat is the crystalline solid as described in U.S. Pat. No. 8,088,803. In some embodiments, the solid form is substantially anhydrous (e.g., contains less than about 1% water, less than about 0.5% water, less than about 1.5% water, less than about 2% water). For example, the water content is determined by Karl Fischer titration. In some embodiments, the solid form is characterized by a melting point of, or a DSC endotherm centered at, about 162 to about 166° C. In some embodiments, the solid form is characterized by a melting point of, or a DSC endotherm centered at, about 164° C. In some embodiments, the solid form has a weight loss of 0.3%, heating from 20° C. to 150° C. at a heating rate of 10° C./min.

In further embodiments, the solid form has at least one, two or three XRPD peaks, in terms of 2-theta, selected from about 18.4°, about 18.9°, about 21.8°, about 23.9°, about 29.2°, and about 38.7°.

In some embodiments, the crystalline form has one or more of the peaks from the list of 2-theta peaks provided in table below.

| 2-Theta | Height | H % |
|---|---|---|
| 3.9 | 74 | 1.1 |
| 7.2 | 119 | 1.8 |
| 13.4 | 180 | 2.8 |
| 14.0 | 150 | 2.3 |
| 15.9 | 85 | 1.3 |
| 18.4 | 903 | 13.9 |
| 18.9 | 1469 | 22.7 |
| 21.3 | 519 | 8 |
| 21.8 | 6472 | 100 |
| 22.7 | 516 | 8 |
| 23.9 | 2515 | 38.9 |
| 24.8 | 804 | 12.4 |
| 25.3 | 182 | 2.8 |
| 27.4 | 476 | 7.4 |
| 28.6 | 354 | 5.5 |
| 29.2 | 1767 | 27.3 |
| 29.9 | 266 | 4.1 |
| 30.6 | 773 | 11.9 |
| 31.2 | 379 | 5.8 |
| 31.6 | 291 | 4.5 |
| 32.7 | 144 | 2.2 |
| 33.5 | 221 | 3.4 |
| 36.4 | 469 | 7.2 |
| 37.6 | 152 | 2.3 |
| 38.7 | 1381 | 21.3 |
| 41.0 | 153 | 2.4 |
| 42.1 | 382 | 5.9 |
| 43.6 | 527 | 8.1 |
| 44.4 | 1080 | 16.7 |

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 4% of the maximum peak height/intensity. Moreover, instrument variation and other factors can affect the 2-theta values. Thus, peak assignments, such as those reported herein, can vary by plus or minus about 0.2° (2-theta), and the term “substantially” as used in the context of XRPD herein is meant to encompass the above-mentioned variations.

In the same way, temperature readings in connection with DSC, TGA, or other thermal experiments can vary about ±3° C. depending on the instrument, particular settings, sample preparation, etc.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### Preparation of Antibodies and Pharmaceutical Compositions of Antibodies

In certain embodiments, the antibodies that bind to human PD-1 include a human heavy chain and light chain constant region. In certain embodiments, the heavy chain constant region comprises a CH1 domain and a hinge region. In some embodiments, the heavy chain constant region comprises a CH3 domain. If the heavy chain constant region includes substitutions, such substitutions modify the properties of the antibody (e.g., increase or decrease one or more of: Fc receptor binding, antibody glycosylation, the number of cysteine residues, effector cell function, or complement function). In certain embodiments, the antibody is an IgG antibody. In specific embodiments, the antibody is selected from the group consisting of IgG1, IgG2, IgG3, and IgG4.

Antibodies, such as ANTIBODY X, can be made, for example, by preparing and expressing synthetic genes that encode the recited amino acid sequences or by mutating human germline genes to provide a gene that encodes the recited amino acid sequences. Moreover, this antibody and other antibodies that bind to human PD-lcan be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16: 237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three-dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237 (based on Kabat numbering). Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

The antibodies that bind to human PD-1 or human PD-Llcan be in the form of full length antibodies, or in the form of low molecular weight forms (e.g., biologically active antibody fragments or minibodies) of the antibodies that bind to human PD-1 or human PD-L1, e.g., Fab, Fab′, F(ab′)2, Fv, Fd, dAb, scFv, and sc(Fv)2. Other antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an antibody that binds to human PD-1 or human PD-L1, or antigen-binding fragment thereof, and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an antibody that binds to human PD-1 or human PD-L1, or antigen-binding fragment thereof, comprising at least one deamidation site, wherein the pH of the composition is from about 5.0 to about 6.5, such that, e.g., at least about 90% of the antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g. as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e. the neutral amide side chain has been converted to a residue with an overall acidic character.

The term “mixture” as used herein in reference to a composition comprising an antibody that binds to human PD-1 or human PD-L1 or antigen-binding fragment thereof, means the presence of both the desired antibody that binds to human PD-1 or human PD-L1, or antigen-binding fragment thereof, and one or more acidic variants thereof. The acidic variants may comprise predominantly deamidated antibody that binds to human PD-1 or human PD-L1, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

### Antibody Fragments

Antibody fragments (e.g., Fab, Fab′, F(ab′)2, Facb, and Fv) may be prepared by proteolytic digestion of intact antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Plueckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)2 fragments (Carter et al., Bio/Technology, 10:163-167 (1992)). According to another approach, F(ab′)2 fragments can be isolated directly from recombinant host cell culture. Fab and F(ab′) 2 fragment with increased in vivo half-life comprising a salvage receptor binding epitope residues are described in U.S. Pat. No. 5,869,046.

### Minibodies

Minibodies of antibodies that bind to human PD-1 or human PD-Llinclude diabodies, single chain (scFv), and single-chain (Fv)2 (sc(Fv)2).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.\

An scFv is a single-chain polypeptide antibody obtained by linking the VH and VL with a linker (see e.g., Huston et al., Proc. Natl. Acad. Sci. U.S.A, 85:5879-5883 (1988); and Plickthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The H chain V region and L chain V region in an scFv may be derived from any antibody that binds to human PD-1 or human PD-L1, or antigen-binding fragment thereof, described herein.

An sc(Fv)2 is a minibody in which two VHs and two VLs are linked by a linker to form a single chain (Hudson, et al., J. Immunol. Methods, (1999) 231: 177-189 (1999)). An sc(Fv)2 can be prepared, for example, by connecting scFvs with a linker. The sc(Fv)2 of the present disclosure include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order.

### Bispecific Antibodies

Bispecific antibodies are antibodies that have binding specificities for at least two different epitopes. Exemplary bispecific antibodies may bind to two different epitopes of the PD-1 protein. Other such antibodies may combine a PD-1 binding site with a binding site for another protein. Bispecific antibodies can be prepared as full length antibodies or low molecular weight forms thereof (e.g., F(ab′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

The antibodies disclosed herein may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g. ⁹⁰Y, ¹³¹I) fluorescent substances, luminescent substances, haptens, enzymes, metal chelates, drugs, and toxins (e.g., calcheamicin, Pseudomonas exotoxin A, ricin (e.g. deglycosylated ricin A chain)).

In one embodiment, to improve the cytotoxic actions of antibodies that bind to human PD-1 or human PD-L1 and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an antibody that binds to human PD-1 or human PD-L1, or antigen-binding fragment thereof, are modified with a moiety that improves its stabilization and/or retention in circulation, e.g., in blood, serum, or other tissues, e.g., by at least 1.5, 2, 5, 10, or 50 fold. For example, the antibody that binds to human PD-1 or human PD-L1, or antigen-binding fragment thereof, can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the antibody that binds to human PD-1 or human PD-L1, or antigen-binding fragment thereof, can be conjugated to a water-soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 5,156,840).

### Methods of Producing Antibodies

Antibodies may be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QlAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an antibody that binds to human PD-1 or human PD-L1 antibody (e.g., ANTIBODY X) is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

Antibodies with Altered Glycosylation

Different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, phagocytosis and antibody feedback, in addition to effector functions of the antibody (e.g., binding to the complement complex C1, which induces CDC, and binding to FcγR receptors, which are responsible for modulating the ADCC pathway) (Nose and Wigzell, 1983; Leatherbarrow and Dwek, 1983; Leatherbarrow et al., 1985; Walker et al., 1989; Carter et al., 1992, PNAS, 89: 4285-4289).

Accordingly, another means of modulating effector function of antibodies includes altering glycosylation of the antibody constant region. Altered glycosylation includes, for example, a decrease or increase in the number of glycosylated residues, a change in the pattern or location of glycosylated residues, as well as a change in sugar structure(s). The oligosaccharides found on human IgGs affects their degree of effector function (Raju, T. S. BioProcess International April 2003. 44-53); the microheterogeneity of human IgG oligosaccharides can affect biological functions such as CDC and ADCC, binding to various Fc receptors, and binding to Clq protein (Wright A. & Morrison S L. TIBTECH 1997, 15 26-32; Shields et al. J Biol Chem. 2001 276(9):6591-604; Shields et al. J Biol Chem. 2002; 277(30):26733-40; Shinkawa et al. J Biol Chem. 2003 278(5):3466-73; Umana et al. Nat Biotechnol. 1999 February; 17(2): 176-80). For example, the ability of IgG to bind C1q and activate the complement cascade may depend on the presence, absence or modification of the carbohydrate moiety positioned between the two CH2 domains (which is normally anchored at Asn297) (Ward and Ghetie, Therapeutic Immunology 2:77-94 (1995).

Glycosylation sites in an Fc-containing polypeptide, for example an antibody such as an IgG antibody, may be identified by standard techniques. The identification of the glycosylation site can be experimental or based on sequence analysis or modeling data. Consensus motifs, that is, the amino acid sequence recognized by various glycosyl transferases, have been described. For example, the consensus motif for an N-linked glycosylation motif is frequently NXT or NXS, where X can be any amino acid except proline. Several algorithms for locating a potential glycosylation motif have also been described. Accordingly, to identify potential glycosylation sites within an antibody or Fc-containing fragment, the sequence of the antibody is examined, for example, by using publicly available databases such as the website provided by the Center for Biological Sequence Analysis (see NetNGlyc services for predicting N-linked glycosylation sites and NetOGlyc services for predicting O-linked glycosylation sites).

In vivo studies have confirmed the reduction in the effector function of aglycosyl antibodies. For example, an aglycosyl anti-CD8 antibody is incapable of depleting CD8-bearing cells in mice (Isaacs, 1992 J. Immunol. 148: 3062) and an aglycosyl anti-CD3 antibody does not induce cytokine release syndrome in mice or humans (Boyd, 1995 supra; Friend, 1999 Transplantation 68:1632). Aglycosylated forms of the PD-1 antibody also have reduced effector function.

Importantly, while removal of the glycans in the CH2 domain appears to have a significant effect on effector function, other functional and physical properties of the antibody remain unaltered. Specifically, it has been shown that removal of the glycans had little to no effect on serum half-life and binding to antigen (Nose, 1983 supra; Tao, 1989 supra; Dorai, 1991 supra; Hand, 1992 supra; Hobbs, 1992 Mol. Immunol. 29:949).

The antibodies that bind to human PD-1 or human PD-L1 of the present disclosure may be modified or altered to elicit increased or decreased effector function(s) (compared to a second PD-1-specific antibody). Methods for altering glycosylation sites of antibodies are described, e.g., in U.S. Pat. Nos. 6,350,861 and 5,714,350, WO 05/18572 and WO 05/03175; these methods can be used to produce antibodies of the present disclosure with altered, reduced, or no glycosylation.

### Solid Tumors and Cancers

The methods described herein involve the treatment of cancers, preferably solid tumors.

In some embodiments, the solid tumor is selected from skin cancer, lung cancer, lymphoma, sarcoma, bladder cancer, cancer of the ureter, urethra, and urachus, gastric cancer, cervical cancer, liver cancer, breast cancer, renal cancer, squamous cell carcinoma, colorectal cancer, endometrial cancer, anal cancer, and a tumor with microsatellite instability-high (MSI-H), mismatch repair deficient (dMMR) and DNA polymerase c exonuclease domain mutation positive disease.

In some embodiments, the solid tumor is selected from cholangiocarcinoma, melanoma, non-small cell lung cancer, small cell lung cancer, Hodgkin's lymphoma, urothelial carcinomagastric cancer, hepatocellular carcinoma, Merkel cell carcinoma, triple-negative breast cancer, renal cell carcinoma, squamous cell carcinoma of the head and neck, and colorectal cancer.

In some embodiments, the solid tumor is microsatellite-stable (MSS). In some embodiments, the solid tumor is PD-L1 positive. In some embodiments, the solid tumor is microsatellite-stable (MSS) and PD-L1 positive. In some embodiments, the solid tumor is endometrial cancer (e.g., endometrial carcinoma). In some embodiments, the solid tumor is bladder cancer (e.g., non-muscle invasive bladder cancer, such as Bacillus Calmette-Guerin unresponsive non-muscle invasive bladder cancer).

Examples of cancers that are treatable using the treatment methods and regimens of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the ureter, urethra, and urachus, gastric cancer, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The methods of the present disclosure are also useful for the treatment of metastatic cancers, especially metastatic cancers that express PD-L1.

In some embodiments, the cancer is endometrial cancer. In some embodiments, the endometrial cancer is microsatellite-stable (MSS). In some embodiments, the endometrial cancer is PD-L1 positive. In some embodiments, the endometrial cancer is microsatellite-stable (MSS) and PD-L1 positive. In some embodiments, the endometrial cancer is metastatic endometrial cancer. In some embodiments, the endometrial cancer is metastatic, microsatellite-stable (MSS), and PD-L1 positive endometrial cancer (e.g., a metastatic, microsatellite-stable (MSS), and PD-L1 positive endometrial carcinoma).

In some embodiments, the present application provides a method of treating microsatellite-stable (MSS), PD-L1 positive endometrial cancer (e.g., microsatellite-stable (MSS), PD-L1 positive endometrial carcinoma) in a patient, comprising administering to said patient:

(i) epacadostat, or a pharmaceutically acceptable salt thereof, at a dose of about 600 mg on a free base basis BID; and

(ii) an antibody, or an antigen-binding fragment thereof, that binds to human PD-1, wherein the antibody comprises (ii-1) a variable heavy (VH) domain comprising VH complementarity determining region (CDR)1, VH CDR2, and VH CDR3; and (ii-2) a variable light (VL) domain comprising VL CDR1, VL CDR2, and VL CDR3; wherein:

(a) the VH CDR1 comprises the amino acid sequence SYWMN (SEQ ID NO:6);

(b) the VH CDR2 comprises the amino acid sequence VIHPSDSETWLDQKFKD (SEQ ID NO:7);

(c) the VH CDR3 comprises the amino acid sequence EHYGTSPFAY (SEQ ID NO:8);

(d) the VL CDR1 comprises the amino acid sequence RASESVDNYGMSFMNW (SEQ ID NO:9);

(e) the VL CDR2 comprises the amino acid sequence AASNQGS (SEQ ID NO:10); and

(f) the VL CDR3 comprises the amino acid sequence QQSKEVPYT (SEQ ID NO:11);

wherein the antibody is administered at a fixed dose of about 375 mg once every three weeks or about 500 mg once every four weeks. In some embodiments, the microsatellite-stable (MSS), PD-L1 positive endometrial cancer, is metastatic microsatellite-stable (MSS), PD-L1 positive endometrial cancer.

In some embodiments, the cancer is bladder cancer. In some embodiments, the bladder cancer is non-muscle invasive bladder cancer (e.g., Bacillus Calmette-Guerin unresponsive non-muscle invasive bladder cancer).

In some embodiments, the present application provides a method of treating non-muscle invasive bladder cancer in a patient, comprising administering to said patient:

(i) epacadostat, or a pharmaceutically acceptable salt thereof, at a dose of about 600 mg on a free base basis BID; and

(ii) an antibody, or an antigen-binding fragment thereof, that binds to human PD-1, wherein the antibody comprises (ii-1) a variable heavy (VH) domain comprising VH complementarity determining region (CDR)1, VH CDR2, and VH CDR3; and (ii-2) a variable light (VL) domain comprising VL CDR1, VL CDR2, and VL CDR3; wherein:

(a) the VH CDR1 comprises the amino acid sequence SYWMN (SEQ ID NO:6);

(b) the VH CDR2 comprises the amino acid sequence VIHPSDSETWLDQKFKD (SEQ ID NO:7);

(c) the VH CDR3 comprises the amino acid sequence EHYGTSPFAY (SEQ ID NO:8);

(d) the VL CDR1 comprises the amino acid sequence RASESVDNYGMSFMNW (SEQ ID NO:9);

(e) the VL CDR2 comprises the amino acid sequence AASNQGS (SEQ ID NO:10); and

(f) the VL CDR3 comprises the amino acid sequence QQSKEVPYT (SEQ ID NO:11);

wherein the antibody is administered at a fixed dose of about 375 mg once every three weeks or about 500 mg once every four weeks.

In some embodiments, the bladder cancer is Bacillus Calmette-Guerin unresponsive non-muscle invasive bladder cancer (i.e., BCG-unresponsive non-muscle invasive bladder cancer). In some embodiments, the bladder cancer is high risk BCG-unresponsive non-muscle invasive bladder cancer. In some embodiments, the bladder cancer is high risk BCG-unresponsive non-muscle invasive bladder cancer with carcinoma in situ (CIS) (e.g., with or without papillary tumors). In some embodiments, the patient having the non-muscle invasive bladder cancer is ineligible for or elected not to undergo cystectomy.

In some embodiments, the cancers treatable with methods of the present disclosure include tumors with microsatellite instability-high (MSI-H), mismatch repair deficient (dMMR) or DNA polymerase c exonuclease domain mutation positive disease.

In some embodiments, the cancer has a ratio of indoleamine-2,3-dioxygenase (IDO) to tryptophan-2,3-dioxygenase (TDO) of at least 10.

In some embodiments, the cancer has a ratio indoleamine-2,3-dioxygenase-hi (IDOhi) to tryptophan-2,3-dioxygenase-low (TDOlow) of at least 50%.

In some embodiments, the cancer is cervical cancer.

In some embodiments, the cancer is renal cancer.

In some embodiments, the cancer kidney renal clear cell carcinoma.

In some embodiments, the cancer cancer is lung cancer.

In some embodiments, the cancer adenocarcinoma of the lung.

In some embodiments, the cancer is squamous cell carcinoma of the lung.

In some embodiments, the cancer is non-small cell lung cancer.

In some embodiments, the cancer is head and neck cancer.

In some embodiments, the cancer is head and neck squamous cell carcinoma.

In some embodiments, cancers treatable with methods of the present disclosure include melanoma (e.g., metastatic malignant melanoma), renal cancer (e.g. clear cell carcinoma), prostate cancer (e.g. hormone refractory prostate adenocarcinoma), breast cancer, colon cancer, lung cancer (e.g. non-small cell lung cancer and small cell lung cancer), squamous cell head and neck cancer, urothelial cancer (e.g. bladder) and cancers with high microsatellite instability (MSIhigh). Additionally, the disclosure includes refractory or recurrent malignancies whose growth may be inhibited using the methods of the disclosure.

In some embodiments, cancers that are treatable using the methods of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CIVIL), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the treatment methods and regimens of the present disclosure include, but are not limited to, cholangiocarcinoma, bile duct cancer, biliary tract cancer, triple negative breast cancer, rhabdomyosarcoma, small cell lung cancer, leiomyo sarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, diseases and indications that are treatable using the treatment methods and regimens of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), post-essential thrombocythemia myelofibrosis, post-polycythemia vera myelofibrosis, post-polycythemia vera/essential thrombocythemia myelofibrosis and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, Askin's tumor, osteosarcoma, rhabdomyosarcoma, angiosarcoma, fibrosarcoma, liposarcoma, myxoma, rhabdomyoma, rhabdosarcoma, fibroma, lipoma, harmatoma, teratoma, sarcoma botryoides, chondrosarcoma, malignant hemangioendothelioma, malignant schwannoma, alveolar soft part sarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, gastrointestinal stromal tumor (GIST), hemangiopericytoma, hemangio sarcoma, Kaposi's sarcoma, leiomyosarcoma, lymphangiosarcoma, lymphosarcoma, malignant peripheral nerve sheath tumor (MPNST), neurofibrosarcoma, synovial sarcoma, and undifferentiated pleomorphic sarcoma.

Exemplary lung cancers include non-small cell lung cancer (NSCLC) (e.g., squamous cell NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers include cancers of the esophagus (carcinoma, squamous cell carcinoma, adenocarcinoma, leiomyosarcoma, lymphoma), stomach (carcinoma, lymphoma, leiomyosarcoma, adenocarcinoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer (e.g., colorectal adenocarcinoma).

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma). In some embodiments, the cancer is a urological cancer (e.g., papilliary kidney carcinoma, testicular germ cell cancer, chromophobe renal cell carcinoma, clear cell renal carcinoma, or prostate adenocarcinoma).

Exemplary liver cancers include hepatoma (hepatocellular carcinoma), cholangiocarcinoma, hepatoblastoma, angio sarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, serous adenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma (e.g., cutaneous squamous cell carcinoma), Kaposi's sarcoma, moles dysplastic nevi, lipoma, angioma, dermatofibroma, and keloids. In some embodiments, diseases and indications that are treatable using the treatment methods and regimens of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

In some embodiments, diseases and indications that are treatable using the treatment methods and regimens of the present disclosure include, but are not limited to an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

In some embodiments, the treatment methods and regimens of the present disclosure cancers selected from, but not limited to, is colorectal cancer, hepatocellular carcinoma, glioma, kidney cancer, breast cancer, multiple myeloma, bladder cancer, neuroblastoma; sarcoma, non-Hodgkin's lymphoma, non-small cell lung cancer, ovarian cancer, pancreatic cancer, a rectal cancer, acute myeloid leukemia (AML), chronic myelogenous leukemia (CML), acute B lymphoblastic leukemia (B-ALL), chronic lymphocytic leukemia (CLL), hairy cell leukemia (HCL), blastic plasmacytoid dendritic cell neoplasm (BPDCN), non-Hodgkin's lymphomas (NHL), including mantel cell leukemia (MCL), and small lymphocytic lymphoma (SLL), Hodgkin's lymphoma, systemic mastocytosis, and Burkitt's lymphoma.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the IDO enzyme with epacadostat includes the administration of epacadostat to an individual or patient, such as a human, having IDO, as well as, for example, introducing epacadostat into a sample containing a cellular or purified preparation containing the IDO enzyme.

As used herein, the term “subject”, “individual” or “patient,” used interchangeably, refers to any animal, including mammals, preferably mice, rats, other rodents, rabbits, dogs, cats, swine, cattle, sheep, horses, or primates, and most preferably humans.

As used herein, the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” refers to preventing a disease, condition or disorder in an individual who may be predisposed to the disease, condition or disorder but does not yet experience or display the pathology or symptomatology of the disease.

### Squamous Cell Carcinoma of the Anal Canal

Squamous cell carcinoma of the anal canal (SCAC) accounts for almost 3% of digestive system cancers and is increasing in frequency due to its association with HPV and HIV infection. Although most patients have localized disease, systemic metastases will develop in approximately 25% of patients, and 5-year survival is poor in these individuals. Salvage chemotherapy with platinum-based regimens is an accepted standard of care; however, responses are not durable, and progression-free and overall survival after these treatments is measured only in months. There are no accepted salvage treatments for patients who progress after first-line chemotherapy.

### Merkel Cell Carcinoma

Merkel cell carcinoma is a rare, aggressive, cutaneous malignancy attributed to multiple factors, such as Merkel cell polyomavirus, UV irradiation, and immunosuppression. This disease typically is found in older adults with light skin types and has a poor prognosis with lower survival rates compared with other skin malignancies. Surgery and/or radiation therapy are indicated and potentially curative for local-regional disease and relapse is common.

The 5-year survival rates for patients with MCC are 75%, 59%, and 25% for primary localized tumors, tumors with regional lymph node metastases (or local recurrences), and tumors with distant metastases, respectively. More than 30% of patients will develop distant metastatic disease, and the 5-year survival rate for these patients is only approximately 10%.

Historically, metastatic MCC has been treated with chemotherapy regimens similar to those used for small cell lung cancer. Platinum-based chemotherapy provides high initial response rates that are of short duration. No survival advantage has ever been demonstrated for chemotherapy in this disease. Chemotherapy is also associated with risk of severe toxicity and toxic death, particularly among older patients.

### Endometrial Cancer

Endometrial cancer is the fourth most common cancer to affect American women with an estimate of 60,050 new cases diagnosed; an estimated 10,470 endometrial cancer related deaths will occur, making it the sixth most common cancer related deaths to affect American women. Globally, it is the fourth most common cause of cancer related death among women. Endometrial cancer is the most common gynecologic malignancy to afflict women, with adenocarcinoma being the most common histology. Cancers diagnosed at an early stage offer good prognosis with curative options of surgery and/or radiation, but aggressive late stage cancers have limited curative therapeutic options, with five year survivals ranging from 20-60%. Standard treatments for locally advanced or metastatic cancers include systemic treatments like hormonal therapy, single agent chemotherapy, such as doxorubicin, or platinum based combination chemotherapy regimens, such as carboplatin and docetaxel. Given the poor long term prognosis for these patients, additional and newer treatments are necessary.

### Pharmaceutical Compositions

In some embodiments, the compound, epacadostat, can be formulated as part of a pharmaceutical composition. In some embodiments, the antibody that binds to human PD-1 or human PD-L1 can be formulated as part of a pharmaceutical composition. The pharmaceutical compositions comprising the compound, and the antibody that binds to human PD-1 or human PD-L1 or antigen-binding fragment thereof described herein can be formulated as pharmaceutical compositions for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977)J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the antibody that binds to human PD-1 or human PD-L1, or antigen-binding fragment thereof, may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In some embodiments, the compound is formulated as part of a pharmaceutical composition, further comprising at least one excipient.

In some embodiments, in making the compositions provided herein, the compound is mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In some embodiments, the pharmaceutical compositions described herein is in the form of tablets.

In preparing a formulation, the compound can be milled to provide the appropriate particle size prior to combining with the other ingredients. In some embodiments, the compound can be milled to a particle size of less than 200 mesh. In some embodiments, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions provided herein can be formulated so as to provide quick, sustained or delayed release of the active ingredient after administration to the patient by employing procedures known in the art.

The compositions can be formulated in a unit dosage form. The term “unit dosage forms” refers to physically discrete units suitable as unitary dosages for human subjects and other mammals, each unit containing a predetermined quantity of the compound calculated to produce the desired therapeutic effect (e.g., the desired PK profile), in association with a suitable pharmaceutical excipient.

In certain embodiments, for preparing solid compositions such as tablets, the compound is mixed with a pharmaceutical excipient to form a solid pre-formulation composition containing a homogeneous mixture of the compound. When referring to these pre-formulation compositions as homogeneous, the compound is typically dispersed evenly throughout the composition so that the composition can be readily subdivided into equally effective unit dosage forms such as tablets, pills and capsules. This solid pre-formulation is then subdivided into unit dosage forms.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compositions described herein can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

In some embodiments, compositions described herein are sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

### Combination Therapy

I. Cancer therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, CDK2, and CDK4/6 kinase inhibitors such as, for example, those described in WO 2006/056399 can be used in combination with the treatment methods and regimens of the present disclosure for treatment of cancers and solid tumors. Other agents such as therapeutic antibodies can be used in combination with the treatment methods and regimens of the present disclosure for treatment of cancers and solid tumors. The one or more additional pharmaceutical agents can be administered to a patient simultaneously or sequentially.

The treatment methods as disclosed herein can be used in combination with one or more other enzyme/protein/receptor inhibitors therapies for the treatment of diseases, such as cancer and other diseases or disorders described herein. For example, the treatment methods and regimens of the present disclosure can be combined with one or more inhibitors of the following kinases for the treatment of cancer: Akt1, Akt2, Akt3, BCL2, CDK2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFβR, PI3K (alpha, beta, gamma, delta, and multiple or selective), CSF1R, KIT, FLK-II, KDR/FLK-1, FLK-4, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Non-limiting examples of inhibitors that can be combined with the treatment methods and regimens of the present disclosure for treatment of cancer include an FGFR inhibitor (FGFR1, FGFR2, FGFR3 or FGFR4, e.g., pemigatinib (INCY54828), INCB62079), an EGFR inhibitor (also known as ErB-1 or HER-1; e.g. erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g. olaparib, rucaparib, veliparib or niraparib), a JAK inhibitor (JAK1 and/or JAK2, e.g., ruxolitinib, baricitinib, itacitinib (INCB39110), an LSD1 inhibitor (e.g., INCB59872 and INCB60003), a TDO inhibitor, a PI3K-delta inhibitor (e.g., INCB50465 and INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer), an adenosine receptor antagonist (e.g., A2a/A2b receptor antagonist), an HPK1 inhibitor, a chemokine receptor inhibitor (e.g. CCR2 or CCRS inhibitor), a SHPT/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), or combinations thereof.

In some embodiments, the treatment methods described herein are combined with administration of a PI3Kδ inhibitor. In some embodiments, the treatment methods described herein are combined with administration of a JAK inhibitor. In some embodiments, the treatment methods described herein are combined with administration of a JAK1 or JAK2 inhibitor (e.g., baricitinib or ruxolitinib). In some embodiments, the treatment methods described herein are combined with administration of a JAK1 inhibitor. In some embodiments, the treatment methods described herein are combined with administration of a JAK1 inhibitor, which is selective over JAK2.

Example antibodies that can be administered in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be administered to a patient in combination with the treatment methods of the present disclosure and are presented as a non-limiting list: a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, IRESSA™(gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Smll, fludarabine, pentostatin, triapine, didox, trimidox, amidox, 3-AP, and MDL-101,731.

The treatment methods and regimens of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteosome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the treatment methods of the disclosure can be used in combination with a chemotherapeutic in the treatment of cancer, and may improve the treatment response as compared to the response to the chemotherapeutic agent alone, without exacerbation of its toxic effects. In some embodiments, the treatment methods of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining treatment methods of the present disclosure with an additional agent.

The agents can be combined with the epacadostat and/or antibody that binds to human PD-1 or human PD-L1, or antigen-binding fragment thereof, of the present treatment methods in a single or continuous dosage form, or the agents can be administered simultaneously or sequentially as separate dosage forms.

In some embodiments, a corticosteroid such as dexamethasone is administered to a patient in combination with the treatment methods of the disclosure where the dexamethasone is administered intermittently as opposed to continuously.

The treatment methods described herein can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The treatment methods described herein can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the treatment methods and regimens of the present disclosure can be used in combination with tumor specific antigen such as heat shock proteins isolated from tumor tissue itself. In some embodiments, the treatment methods described herein can be combined with dendritic cells immunization to activate potent anti-tumor responses.

The treatment methods and regimens of the present disclosure can be used in combination with bispecific macrocyclic peptides that target Fe alpha or Fe gamma receptor-expressing effectors cells to tumor cells. The treatment methods and regimens of the present disclosure can also be combined with macrocyclic peptides that activate host immune responsiveness.

In some further embodiments, the treatment methods of the disclosure are combined with administration of other therapeutic agents to a patient prior to, during, and/or after a bone marrow transplant or stem cell transplant. The treatment methods and regimens of the present disclosure can be used in combination with bone marrow transplant for the treatment of a variety of tumors of hematopoietic origin.

When more than one pharmaceutical agents is administered to a patient, as discussed in any of the above embodiments, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

### II. Immune-Checkpoint Therapies

Treatment methods of the present disclosure can be used in combination with administration of one or more immune checkpoint inhibitors or agonists (e.g., antibodies or small molecules) for the treatment of diseases, such as cancer. Exemplary immune checkpoint molecules include CBL-B, CD20, CD28, CD40, CD70, CD122, CD96, CD73, CD47, CDK2, GITR, CSF1R, JAK, PI3K-delta, PI3K-gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, and VISTA. In some embodiments, the immune checkpoint molecule is a stimulatory checkpoint molecule selected from CD27, CD28, CD40, ICOS, OX40, GITR and CD137 (4-1BB). In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is an inhibitor of KIR, TIGIT, LAIR1, CD160, 2B4 or TGFR beta.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor is MCLA-145.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an agonist of GITR. In some embodiments, the agonist of GITR is TRX518, MK-4166, INCAGN1876, MK-1248, AMG228, BMS-986156, GWN323, MEDI1873, or MEDI6469.

In some embodiments, the agonist of an immune checkpoint molecule is an agonist of OX40, e.g., OX40 agonist antibody or OX40L fusion protein. In some embodiments, the OX40 agonist antibody is INCAGN01949, MEDI0562 (tavolimab), MOXR-0916, PF-04518600, GSK3174998, BMS-986178, or 9B12. In some embodiments, the agonist of an OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The treatment methods and regimens of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of TDO, or arginase.

As provided throughout, the additional compounds, inhibitors, agents, etc. can be combined with the present compound in a single or continuous dosage form, or they can be administered simultaneously or sequentially as separate dosage forms.

### Labeled Compound

Another aspect of the present disclosure relates to labeled epacadostat (radio-labeled, fluorescent-labeled, isotopically-labeled, etc.) that would be useful not only in imaging techniques but also in assays, both in vitro and in vivo, for localizing and quantitating IDO1 in tissue samples, including human.

The present disclosure further includes isotopically-labeled epacadostat. An “isotopically” or “radio-labeled” compound is epacadostat, where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms can be optionally substituted with deuterium atoms.

One or more constituent atoms of epacadostat can be replaced or substituted with isotopes of the atoms in natural or non-natural abundance. In some embodiments, epacadostat includes at least one deuterium atom. For example, one or more hydrogen atoms in a compound presented herein can be replaced or substituted by deuterium. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et al. J. Med. Chem. 2011, 54, 201-210; R. Xu et al. J. Label Compd. Radiopharm. 2015, 58, 308-312). In particular, substitution at one or more metabolism sites may afford one or more of the therapeutic advantages.

It is understood that a “radio-labeled” or “labeled compound” is a compound that has incorporated at least one radionuclide. In some embodiments, the radionuclide is selected from the group consisting of ³H and ¹⁴C. In some embodiments, the radionuclide is selected from the group consisting of ¹¹C, ¹⁸F, ⁷⁵Br, ⁷⁶Br, and ⁷⁷Br.

### Kits

The present disclosure also includes pharmaceutical kits useful, for example, in the treatment cancers and solid tumors referred to herein, which include one or more containers containing a pharmaceutical composition described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The following are examples of the practice of the invention. They are not to be construed as limiting the scope of the invention in any way.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1. Phase 1b Study of Epacadostat in Combination with ANTIBODY X

### General Study Design

The study is an open-label, nonrandomized, multicenter, Phase 1b study with independent treatment groups. The study consists of 2 parts: 1) dose escalation to find the maximum tolerated dose (MTD)/recommended phase 2 dose (RP2D) of the combination of ANTIBODY X and epacadostat, and 2) expansion at the chosen dose to further explore safety and preliminary evidence of clinical activity.

For dose escalation, a Bayesian optimal interval (BOIN) design with a cohort size of approximately 3 evaluable participants is used. The target rate of dose-limiting toxicities (DLTs) is assumed to be 30% for each combination. At each dose level, a maximum of 9 participants are enrolled. Dose levels for the combination of ANTIBODY X with epacadostat are given in Table 1.

TABLE 1
Dose Levels for ANTIBODY X in Combination With Epacadostat

| Cohort | Dose of ANTIBODY X | Dose of Epacadostat |
|---|---|---|
| −1 | 500 mg Q4W | 50 mg BID |
| 1 (starting dose) | 500 mg Q4W | 100 mg BID |
| 2 | 500 mg Q4W | 300 mg BID |
| 3 | 500 mg Q4W | 400 mg BID |
| 4 | 500 mg Q4W | 600 mg BID |
| 5 | 500 mg Q4W | 900 mg BID |
| 6 | 500 mg Q4W | 1200 mg BID |

The treatment groups enroll in parallel in a nonrandomized fashion with participants assigned to open cohorts by the sponsor or designee. Priority is given to open dose-escalation cohorts. If more than one dose-expansion cohort is available, participants are assigned in alternating fashion with consideration of available data regarding the combinations in the participant's tumor type until enrollment is complete. Based on emerging pharmacokinetic (PK) or pharmacodynamic data (including the results of exploratory immunoassays), additional dose levels or schedules may be explored or some of the dose-escalation cohorts may be expanded or not be opened. Intermediate dose levels or alternative dose schedules may be explored to collect additional safety, PK, and pharmacodynamic data. Also, an intermediate dose level may be explored if the higher dose level exceeds the MTD.

Participants in the dose-escalation cohorts are observed for 28 days for occurrence of DLTs. Participants receiving ANTIBODY X in combination with epacadostat must receive at least 75% of the oral doses to be evaluable for DLT.

Once the RP2D of the combination is determined, an ongoing participant receiving lower doses may be permitted to escalate to the RP2D with approval of the medical monitor if the participant meets Protocol eligibility criteria at the time of escalation, has tolerated the current doses without drug-related toxicity ≥Grade 2, and the investigator determines the participant may potentially benefit from the higher dose.

The MTD is defined as the highest dose at which less than approximately one-third of the participants have a DLT. Dose-limiting toxicities occurring during the first 28 days of treatment guide dose escalation and determination of the MTD and RP2D. In addition, participants with late-onset safety events meeting the definition of DLT or those who had intolerable, lower grade persistent toxicity determined to be attributable to either study drug (e.g., Grade 2 peripheral neuropathy) are considered in the selection of each combination RP2D. The RP2D can be selected from any of the available dose levels that do not exceed the MTD. If an MTD is not reached, the RP2D is selected from the available doses based on safety, pharmacokinetics (PK), and translational data.

Baseline tumor biopsy samples are acquired for all participants. Treatment cycles are 28 days unless otherwise noted. At the beginning of each treatment cycle after Cycle 1, the participant must meet the following criteria:
(i) Hemoglobin ≥8 g/dL
(ii) ANC≥1.0×10⁹/L
(iii) Platelet count ≥75×10⁹/L
(iv) ALT/AST/bilirubin ≤Grade 2
(v) Resolution of all immune-related treatment emergent adverse events (TEAEs) to ≤Grade 1 (with the exception of hyperglycemia [allowed to Grade 2] and endocrinopathy that is controlled on hormonal replacement)
(vi) Resolution of all non-immune-related TEAEs to ≤Grade 1 or baseline (with the exception of Grade 2 alopecia). Transient asymptomatic laboratory elevations ≤Grade 3 do not require dose interruption if the participant is asymptomatic and if the elevation is clinically insignificant and has been discussed with the medical monitor.

Treatment duration on study is up to 2 years in the absence of clinical progression or intolerable toxicity. The study will end once the last participant in each treatment group has been followed for approximately 6 months.

Participants are eligible to be included in the study only if all of the following criteria apply:
Ability to comprehend and willingness to sign a written ICF for the study.
Adult men and women 18 years of age or older (or as applicable per local country requirements).
Participants with histologically proven, locally advanced unresectable or metastatic solid tumors for whom no approved therapy with demonstrated clinical benefit is available or participants who are intolerant to or have declined standard therapy.
Measurable or nonmeasurable tumor lesions per RECIST v 1.1. (Note: Participants enrolled in the dose-escalation cohorts must have at least 1 lesion that can be biopsied).
Willing to provide fresh or archival tumor tissue for correlative studies.
Eastern Cooperative Oncology Group (ECOG) performance status 0 to 1.
Willingness to avoid pregnancy or fathering children based on specific criteria.

Participants are excluded from the study if any of the following criteria apply:
Receipt of anticancer therapy within 21 days of the first administration of study treatment, with the exception of localized radiotherapy.
Toxicity of prior therapy that has not recovered to ≤Grade 1 or baseline (with the exception of alopecia and anemia not requiring transfusional support).
Participants with laboratory values at screening defined in Table 2.
Active autoimmune disease requiring systemic immunosuppression in excess of physiologic maintenance doses of corticosteroids.
Known active CNS metastases and/or carcinomatous meningitis.
Known additional malignancy that is progressing or requires active treatment, or history of other malignancy within 2 years of study entry with the exception of cured basal cell or squamous cell carcinoma of the skin, superficial bladder cancer, prostate intraepithelial neoplasm, carcinoma in situ of the cervix, or other noninvasive or indolent malignancy, or cancers from which the participant has been disease-free for >1 year, after treatment with curative intent.
Known active hepatitis A, B, or C, as defined by elevated transaminases with the following serology: positivity for hepatitis A virus IgM antibody, anti-hepatitis C virus, anti-hepatitis B core antigen IgG or IgM, or hepatitis B surface antigen in the absence of prior immunization.
Active infections requiring systemic antibiotics.
Any ≥Grade 2 immune-related toxicity while receiving prior immunotherapy.
Known hypersensitivity to any of the study drugs, excipients, or another monoclonal antibody which cannot be controlled with standard measures (eg, antihistamines and corticosteroids).
Participants with impaired cardiac function or clinically significant cardiac disease:
   New York Heart Association Class III or IV cardiac disease, including preexisting clinically significant ventricular arrhythmia, congestive heart failure, or cardiomyopathy
   Unstable angina pectoris ≤6 months before study participation.
   Acute myocardial infarction ≤6 months before study participation.
   Other clinically significant heart disease (ie, ≥Grade 3 hypertension, history of labile hypertension, or poor compliance with an anti-hypertensive regimen) must have recovered (to baseline or ≤Grade 1) from toxicity associated with prior treatment.
Women who are pregnant or breast-feeding.
If participant received major surgery, then they must have recovered adequately from toxicities and/or complications from the intervention before starting study treatment.
Has received a live vaccine within 30 days of the planned start of study treatment.
Evidence of interstitial lung disease or active, noninfectious pneumonitis.
Current use of prohibited medications, including other anticancer therapies, including investigational treatments; immunosuppression in excess of physiologic maintenance corticosteroid doses (with the exception of acute treatment for an AE); white blood cell transfusions; live vaccines during the study and for a duration of 5 half-lives; products containing acetyl-para-aminophenol in excess of 2 g or 2000 mg total daily dose; any MAOI or drug associated with significant MAO inhibitory activity agents is prohibited from 21 days before starting study treatment through 14 days after the final dose of epacadostat has been taken; and coumarin-based anticoagulants.
Any condition that would, in the investigator's judgment, interfere with full participation in the study, including administration of study treatment and attending required study visits; pose a significant risk to the participant; or interfere with interpretation of study data.
Participants may not have a history of serotonin syndrome after receiving 1 or more serotonergic drugs.
Participants who are known to be HIV-positive, unless all of the following criteria are met:
   CD4+ count ≥300/μL.
   Undetectable viral load.
   Receiving highly active antiretroviral therapy.
Participants may not have a history of a gastrointestinal condition (eg, inflammatory bowel disease, Crohn's disease, ulcerative colitis) that may affect drug absorption.

TABLE 2

| | |
|---|---|
| Laboratory | |
| Parameter | Exclusion Criterion |

Hematology

| | | |
|---|---|---|
| a | Platelets | <100 × 10⁹/L |
| b | Hemoglobin | <8 g/L |
| c | ANC | <1.5 × 10⁹/L |

Hepatic

| | | |
|---|---|---|
| d | ALT | >2 × ULN |
| e | AST | >2 × ULN |
| f | Bilirubin | ≥1.5 × ULN unless conjugated bilirubin ≤ ULN |
| | | (conjugated bilirubin only needs to be tested if total |
| | | bilirubin exceeds ULN). If there is no institutional |
| | | ULN, then direct bilirubin must be < 40% of total |
| | | bilirubin |

Renal

| | | |
|---|---|---|
| g | Serum | >1.5 × institutional ULN OR measured or calculated |
| | creatinine | creatinine clearance (glomerular filtration rate can also |
| | | be used in place of creatinine or creatinine clearance) |
| | | <50 mL/min |

Coagulation

| | | |
|---|---|---|
| h | INR or PT | >1.5 × ULN unless on therapeutic anticoagulants |
| i | aPTT | >1.5 × ULN |

Table 3 presents the study treatment information for infused study drug and oral study drug. At visits where oral study drug is administered in the clinic, the oral study drug is administered just before the start of the ANTIBODY X infusion.

TABLE 3

| | | |
|---|---|---|
| Study treatment name: | ANTIBODY X | Epacadostat |
| Dosage formulation: | liquid formulation | 25 mg, 100 mg (both uncoated), |
| | | and 300 mg (coated) tablets |
| Unit dose strength(s)/ | 500 mg Q4W | 50 mg BID |
| dosage level(s): | | 100 mg BID |
| | | 300 mg BID |
| | | 400 mg BID |
| | | 600 mg BID |
| | | 900 mg BID |
| | | 1200 mg BID |
| Route of administration: | IV | PO |
| Administration | IV over 60 (+15) minutes. | BID with water without regard to |
| instructions: | | food except on the mornings of PK |
| | | clinic visits |
| Packaging and labeling: | ANTIBODY X 25 mg/mL will be | Tablets will be packaged in |
| | provided in a glass vial for | high-density polyethylene bottles. |
| | single use. | Each bottle/blister card will be |
| | Each vial will be labeled as | labeled as required per country |
| | required per country requirement. | requirement. |
| Storage: | Upright under refrigeration at 2° C.- | Room temperature, 15° C.-30° C. |
| | 8° C. (36° F.-46° F.) | (59° F.-86° F.) |
| | Protected from light | |

### Dose Limiting Toxicity

A DLT is defined as the occurrence of any of the toxicities listed in Table 4 that are possibly, probably, or definitely due to study treatment occurring from the start of treatment up to and including Day 28. All DLTs will be assessed by the investigator using Common Terminology Criteria for Adverse Events: Version 5 (CTCAE v5) criteria. Participants receiving ANTIBODY X in combination with an oral study drug must receive at least 75% of the oral doses to be evaluable for DLTs. If study treatment is interrupted because of a drug-related toxicity, this will be considered a DLT.

TABLE 4
Definition of Dose-Limiting Toxicity

General
Any death not clearly due to the underlying disease or extraneous causes.
Any Grade 2 toxicity that (in the opinion of the investigator) is potentially life-threatening and
cannot be controlled with standard measures (eg, corticosteroids).
A drug-related AE of any grade observed during the DLT evaluation period that leads to a
continuous drug interruption and that prevents a participant from receiving at least 75% of the
planned cohort-specified doses of study treatment, or is the primary reason for the permanent
discontinuation of study treatment.
Hematologic toxicity
Grade 4 thrombocytopenia or ≥ Grade 3 thrombocytopenia with clinically significant bleeding
(requires hospitalization, transfusion of blood products, or other urgent medical intervention).
≥Grade 4 neutropenia lasting > 5 days or accompanied by fever.
Grade 4 anemia not explained by underlying disease or unrelated illnesses (eg, hemolysis).
Nonhematologic toxicity
Any ≥ Grade 3 nonhematologic toxicity EXCEPT for the following:
Transient (≤72 hours) abnormal laboratory values not requiring management (eg, amylase).
Grade 3 nausea/vomiting or diarrhea < 72 hours with adequate antiemetic and other supportive
care.
Grade 3 fatigue < 1 week.
Asymptomatic changes in lipid profiles or blood glucose.
Alopecia.
Events meeting Hy's Law criteria (defined as an increase in AST or ALT > 3 × ULN and total
bilirubin > 2 × ULN, where no other reason can be found to explain the combination of increases).

At the beginning of each treatment cycle, the participant must meet the treatment continuation criteria noted above before the infusion of ANTIBODY X. If the criteria are not met, study treatment (both study drugs) is interrupted. Participants are withdrawn from the active treatment portion of the study if treatment continuation criteria are not met within 28 days of the scheduled start of a cycle. If either study drug in a combination must be discontinued due to unacceptable toxicity then the participant is withdrawn from both study drugs (ie, study treatment) and enters the follow-up portion of the study.

Dose reductions are not allowed for ANTIBODY X and epacadostat.

### Response Evaluation

To evaluate response in solid tumors, the Response Evaluation Criteria in Solid Tumors (RECIST) v1.1 guidelines are followed. The recommended method for measuring and following tumor burden is determined by a CT scan, which is performed using consistent techniques and facilities. Alternative modalities (e.g., MRI) may be substituted for a CT scan at the discretion of the investigator, provided that the same modality is used throughout the study and the methodology is consistent with RECIST v1.1. Initial tumor imaging is performed within 28 days before the first dose of study treatment. Tumor lesions that are located in a previously irradiated area or in an area subjected to other locoregional therapy are not selected as target lesions. Additionally, it is recommended that tumor lesions selected for biopsy not be selected as target lesions.

Immunotherapeutic agents may produce antitumor effects by potentiating endogenous cancer specific immune responses. The response patterns seen with such an approach may extend beyond the typical time course of responses seen with cytotoxic agents and can manifest a clinical response after an initial increase in tumor burden or even the appearance of new lesions. Standard RECIST v1.1 may not provide a fully accurate response assessment of immunotherapeutic agents and may require participants to be removed from treatment who may otherwise have benefited from further immunotherapy treatment. Therefore, the general principles of a modified version of RECIST v1.1 for immune-based therapeutics, termed iRECIST, is used in the evaluation of participant response in an exploratory capacity in this study. The use of iRECIST accounts for the response patterns of immunotherapies and includes a requirement for the confirmation of progression to rule out or confirm pseudoprogression.

Adverse events are monitored with all serious adverse events (SAEs) being recorded and reported. Clinical laboratory tests are performed, including measurement of kynurenine levels in blood plasma and tumor samples.

Plasma kynurenine levels were measured by an LC-MS/MS method at World Wide Clinical Trials, Inc. Patient samples were obtained pre-dose and at defined times following treatment. The plasma kynurenine levels can be measured substantially as described by Huang, et al., Bioanalysis, 2013; 5(11): 1397-1407.

Kynurenine levels in flash frozen tumor samples will be measured by quantitative mass spectrometry imaging or by LC-MS/MS. Tumor biopsies will be obtained prior to treatment and during week 5 of treatment.

### Follow-Up Analysis

Participants who discontinue study treatment for a reason other than disease progression will move into the disease status follow-up period and should be assessed every 12 weeks±7 days by radiologic imaging to monitor disease status. Efforts can be made to collect information regarding disease status until the start of new anticancer therapy; disease progression; death; the end of the study; and participant is lost to follow-up. Once a participant has received the last dose of study treatment, has confirmed disease progression, or starts a new anticancer therapy, the participant moves into the survival follow-up period and should be contacted by telephone, email, or visit at least every 12 weeks to assess for survival status until death, withdrawal of consent, or the end of the study, whichever occurs first.

### Results

In the study above, two groups of three patients each (Group 1 and Group 2) suffering from solid tumors were given ANTIBODY X (500 mg Q4W) in combination with 100 mg BID or 600 mg BID epacadostat. The patients in Group 1 were administered 100 mg BID epacadostat, while Group 2 were administered 600 mg BID epacadostat. The patients in Group 2 showed increased reductions in plasma kynurenine levels compared to the three patients in Group 1 at day 8 of treatment with 2/3 showing sustained reductions after 5 weeks of treatment. This suggests that higher doses of epacadostat result in higher levels of IDO1 inhibition. This reduction in plasma kynurenine levels with 600 mg BID epacadostat was surprising based on previous clinical trials results with 300 mg BID epacadostat in combination with another anti-PD-1 antibody, pembrolizumab (200 mg/kg Q3W), which showed Iₘₐₓ, Iₘᵢₙ and I_{avg} values of 97%, 76%, and 88%.

The term “Iₘₐₓ” refers to the maximum percentage of the calculated IDO inhibition across all the PK time points. Iₘₐₓ is the maximum or highest percentage of IDO inhibition between the time when the drug is administered to its trough (e.g., the lowest concentration of the drug that is present in the subject). For example, in a twice-daily administration, Iₘₐₓ refers to the highest percentage of IDO inhibition during the period between 0 hour (pre-dose) and 12^{th} hour after dosing.

The term “Iₘᵢₙ” refers to the minimum percentage of the calculated IDO inhibition across all the PK time points. Iₘᵢₙ is the percentage of IDO inhibition at trough (e.g., generally at the 12^{th} hour in a twice-daily administration). For example, Iₘᵢₙ≥50 refers to IDO inhibition is 50% or greater at trough (e.g., at the 12^{th} hour).

The term “I_{avg}” refers to the average percentage of IDO inhibition during the period from which the drug is administered to trough. It is calculated as the area under the inhibition curve over time (AUC) (calculated using a linear trapezoidal method) divided by the dosing interval (e.g., 12 hours for BID dosing).

The calculated Iₘₐₓ, Iₘᵢₙ and I_{avg} values of each subject were summarized as mean±standard deviation (geometric mean) standard statistical calculations for every dose group.

The combination of ANTIBODY X and epacadostat has been assessed in a dose-finding study (INCMGA 0012-102, NCT03059823). 31 participants were treated with the combination of ANTIBODY X 500 mg Q4W and epacadostat at doses of 100 mg, 400 mg, 600 mg, and 900 mg BID. Epacadostat 900 mg BID exceeded the MTD, based on the development of Grade 3 rash in 2 of 3 participants with the third participant developing rash just after the protocol-defined DLT window. Treatment-emergent adverse events (TEAEs) reported in greater than 10% of participants included fatigue, nausea, abdominal pain, pruritus, rash maculo-papular, and diarrhea. Serious adverse events (SAE) occurred in 8 participants (25.8%) however no SAE occurred in >1 participant. Three participants has a dose-limiting toxicity (DLT), all of which were Grade 3 maculo-papular rash (one DLT occurred at the 400 mg BID dose of epacadostat in combination with ANTIBODY X and two occurred at the 900 mg BID dose of epacadostat). Epacadostat 600 mg BID was well-tolerated in combination with ANTIBODY X 500 mg Q4W in the initial cohort of participants and is being further evaluated. In addition, epacadostat 600 mg BID resulted in durable normalization of kynurenine in preliminary observations.

FIG. 1 shows plasma kynurenine results of patients treated with ANTIBODY X in combination with the indicated doses of epacadostat (100 mg BID; 400 mg BID; 600 mg BID; 900 mg BID). Plasma kynurenine was measured pre-treatment (C1D1) and at the indicated visits. FIG. 1 shows that treatment with 600 mg BID resulted in sustained (up to 4 months) decreases in plasma kyn in most patients.

### Example 2. Phase 2 Study of ANTIBODY X in Combination with Epacadostat in Patients with Recurrent or Advanced PD-L1 Positive Microsatellite-Stable Endometrial Cancer

### General Study Design

This is a multicenter, open-label, nonrandomized, Phase 2 study of ANTIBODY X in combination with epacadostat in participants who have advanced or metastatic endometrial cancer that is microsatellite-stable (MSS) and PD-L1 positive and that has progressed on or after platinum-based chemotherapy. Participants will receive ANTIBODY X 500 mg Q4W (IV administration) in combination with epacadostat 600 mg BID (PO administration) for up to 26 cycles. This study will include one interim analysis for futility after 24 participants have been enrolled. Table 5 describes the objectives and endpoints for this study.

TABLE 5

| | |
|---|---|
| Objectives | Endpoints |

Primary

| | |
|---|---|
| To determine the efficacy of ANTIBODY X in | Objective response rate (ORR), defined as the |
| combination with epacadostat in participants with | percentage of participants with best overall |
| advanced or metastatic MSS, PD-L1 positive | response of confirmed complete response (CR) or |
| endometrial cancer. | partial response (PR) determined by independent |
| | central review (ICR) per RECIST v1.1 |

Secondary

| | |
|---|---|
| To further evaluate clinical efficacy of the | Duration of response (DOR), defined as the |
| combination of ANTIBODY X and epacadostat. | time from the first confirmed objective response |
| | (CR or PR) according to RECIST v1.1 (as |
| | determined by ICR) until disease progression or |
| | death due to any cause. |
| | Disease control rate (DCR), defined as the |
| | proportion of participants with best overall |
| | confirmed response of CR or PR, or SD for at |
| | least 24 weeks (as determined by ICR). |

Exploratory

| | |
|---|---|
| To evaluate the pharmacokinetics (PK) of | The PK of ANTIBODY X when given in |
| ANTIBODY X when in combination with | combination with epadadostat (including Cₘₐₓ, |
| epacadostat. | Tₘₐₓ, Cₘᵢₙ, and AUC₀₋ₜ) will be summarized. |
| To evaluate the PK of epacadostat when given in | The PK of epacadostat when given in combination |
| combination with ANTIBODY X. | with ANTIBODY X (including Cₘₐₓ, Tₘₐₓ, Cₘᵢₙ, |
| | and AUC₀₋ₜ) will be summarized. |
| To determine the efficacy of ANTIBODY X in | ORR, defined as the percentage of participants |
| combination with epacadostat by investigator | with best overall response of confirmed CR or |
| assessment in participants with advanced or | PR determined by investigator per RECIST v1.1 |
| metastatic MSS, PD-L1 positive endometrial | and iRECIST |
| cancer. | DOR, defined as the time from the first |
| | confirmed objective response (CR or PR) |
| | according to RECIST v1.1 (as determined by |
| | investigator) and iRECIST until disease |
| | progression or death due to any cause. |
| | DCR, defined as the proportion of participants |
| | with best overall confirmed response of CR or |
| | PR, or SD for at least 24 weeks (as determined |
| | by investigator) according to RECIST v1.1 and |
| | iRECIST. |
| | Progression-free survival (PFS) defined as the |
| | time from the first dose of study treatment until |
| | disease progression (as determined by ICR) or |
| | death due to any cause by ICR according to |
| | RECIST v1.1 and iRECIST. |
| | Overall survival (OS), defined as the time from |
| | the first dose of study treatment until death due |
| | to any cause. |
| To further evaluate clinical efficacy of of the | ORR defined as the percentage of participants |
| combination of ANTIBODY X and epacadostat | with best overall response of confirmed CR or |
| by ICR assessment in participants with advanced | PR determined by ICR according to iRECIST. |
| or metastatic MSS, PD-L1 positive endometrial | DOR, defined as the time from the first |
| cancer. | confirmed objective response (CR or PR) |
| | according to iRECIST as determined by IRC |
| | until disease progression or death due to any |
| | cause. |
| | DCR, defined as the proportion of participants |
| | with best overall confirmed response of CR or |
| | PR, or SD for at least 24 weeks (as determined |
| | by ICR) according to iRECIST |
| | PFS defined as the time from the first dose of |
| | study treatment until disease progression |
| | defined by RECISTv1.1 and iRECIST (as |
| | determined by ICR) or death due to any cause. |

After discontinuation of study treatment, the treatment portion of the study will end, and the participant will enter follow-up. Follow-up consists of 3 parts, safety follow-up, disease status follow-up and survival follow-up. Participants are followed for safety for 90 days after the last dose of study treatment or until they begin a new anticancer therapy, whichever occurs first. Participants who discontinue study treatment for a reason other than disease progression will move into the disease status follow-up period and should continue to be assessed Q8W to monitor disease status until the start of a new anticancer therapy, disease progression, death, the end of the study, or the participant is lost to follow-up.

### Background and Rationale

Blockade of immune inhibitory pathways is emerging as an important therapeutic modality for the treatment of cancer as evidenced by the clinical responses observed with antibodies to PD-1/PD-L1. Although these single agents have antitumor activity, multiple immune inhibitory mechanisms are present concurrently within the tumor microenvironment, suggesting that combination therapies may be required for optimal therapeutic effect (Quezada & Peggs, Br. J. Cancer. 2013, 108:1560-1565). The purpose of this study is to examine the safety and efficacy of ANTIBODY X, a PD-1 inhibitor, in combination with epacadostat, an IDO1 inhibitor, which may improve the therapeutic efficacy of anti-PD-1 monotherapy in patients with PD-L1 positive, MSS endometrial cancer.

Endometrial cancer (EC) is the most common gynecological cancer in developed countries (Colombo et al, Int. J. Gynecol. Cancer 2016, 26:2-30). In 2018, approximately 380,000 new cases of endometrial cancer were diagnosed worldwide and it is estimated that 90,000 women died globally from this disease. It is the sixth most common cancer in women globally (Brey et al, CA Cancer J. Clin. 2018, 68:394-424). Approximately 65,620 new cases and 12,590 deaths from endometrial cancer are expected in the United States in 2020. Two thirds of new cases are diagnosed at early stage. Average age at presentation is 60 years and it is rare in women under 45 years of age. Rates of endometrial cancer have increased over time and in successive generations in many countries across the world, particularly in those with rapid socio-economic transition (Lortet-Tieulent et al, J. Natl. Cancer Inst. 2018, 110:354-361). While the 5 year survival rate is 95% for localized disease, only 17% of women with distant metastatic disease are expected to survive 5 years from diagnosis.

Risk factors for endometrial cancer include increased levels of estrogen (caused by obesity, diabetes, and high-fat diet), early age at menarche, nulliparity, late age at menopause, older age (≥55 years), and tamoxifen use (Van den Bosch et al, Best Pract. Res. Clin. Obstet. Gynaecol. 2012, 26:257-66; Kitchener & Trimble, Int. J. Gynecol. Cancer, 2009, 19:134-140; Dinkelspiel et al, Obstet. Gynecol. Int. 2013, 2013:583891; Obermair et al, Int. J. Cancer, 2010, Dec. 1, 127:2678-2684). Obesity with BMI greater than 30 is responsible for up to 81% of newly diagnosed endometrial cancers (Nevadunsky et al, Obstet. Gynecol. 2014, 124:300-306). The incidence of endometrial cancer is increasing primarily because of increased incidence of obesity and resulting hyperinsulinemia.

Most endometrial cancers are sporadic, but 2-5% of cases are familial and have germline mutations in mismatch repair genes (Lynch et al, Nat. Rev. Cancer, 2015, 15:181-194). Four molecular clusters of EC have been identified in the comprehensive study of 373 ECs through The Cancer Genome Atlas (TCGA) (Kandoth et al, Nature, 2013, 497:67-73). These are: (1) ultramutated/polymerase E (POLE)-mutated; (2) hypermutated/MSI (MSI-H); (3) copy number-low (microsatellite stable[MSS]); and (4) copy number-high. POLE tumors had the best PFS and the copy number-high tumors were the worst. Unfortunately, genome sequencing methods used in TCGA are not suitable for wider clinical application. Localized endometrial cancer can be cured by surgical resection. Systemic therapies are used in more advanced disease. Hormonal therapies are preferred in low grade hormone positive disease that is not rapidly progressive. It is not recommended for patients with visceral and rapidly progressing disease (see Colombo et al, Int. J. Gynecol. Cancer, 2016, 26:2-30). Endometrial cancer is chemo-sensitive and, multi-agent chemotherapy is preferred for metastatic, recurrent, or high-risk disease (Colombo et al, Int. J. Gynecol. Cancer, 2016, 26:2-30; National Comprehensive Care Network. Clinical Practice Guidelines in Oncology. Uterine Neoplasms. Version 3.2019—11 February 2019). Anthracyclines, taxanes and platinum based compounds have been extensively studied in this disease. A combination of carboplatin and paclitaxel is commonly used as first line therapy in advanced EC and has an ORR of approximately 50%, PFS of 13 months and OS of 3 years (Miller et al, Gynecol. Oncol. 2012, 125:771-773; Colombo et al, Int. J. Gynecol. Cancer, 2016, 26:2-30).

Treatment options following failure of first-line chemotherapy are limited (Fleming et al, J. Clin. Oncol. 2015, 33:3535-3540). After failure of primary chemotherapy, there is no established active second line agent in this disease. Paclitaxel has the highest RR of 25% in patients previously treated with combination of cisplatinum and doxorubicin. The RR with docetaxel in patients treated with paclitaxel in first line therapy is only 8%. The 5 year survival for advanced/recurrent measurable disease after second line therapy is <10% (Moxeley et al, The Oncologist, 2010, 15:1026-1033; Dizon et al, J. Clin. Oncol. 2009, 27:3104-3108; and Garcia et al, Gynecol. Oncol. 2008, 111:22-26). Everolimus plus letrozole and bevacizumab have also shown modest activity in small uncontrolled trials as have PD-1 inhibitors monotherapy and in combination with other therapies in tumors that were not selected for abnormalities in DNA repair (Ott et al, J. Immunother. Cancer 2017, 5:16; and Oaknin et al, Gynecol. Oncol. 2019, 154(1 suppl):Abstract 33). MMR deficiency, in particular, has been associated with resistance to the commonly used chemotherapy agents (Guillotin & Martin, Exper. Cell Res. 2014, 329:110-115). In approximately 25-30% of EC, the tumors are MMR-deficient or MSI-H (Murali et al, Lancet Oncol. 2014, June; 15(7):e268-278; Karamurzin and Rutgers, Int. J. Gynecol. Pathol. 2009, 28:239-255). Promising clinical activity with immunotherapy based approaches has been seen in tumors characterized by abnormalities in DNA repair (eg, MSI-H, dMMR, or POLE ultra-mutated) that are associated with high neoantigen load (Mittica et al, Oncotarget, 2017, 8:90532-90544; Brooks et al, CA Cancer J. Clin. 2019, 69:258-279; and Di Tucci et al, J. Gynecol. Oncol. 2019, 30:e46). Pembrolizumab has been shown to be effective in treatmenmt of MMR deficient tumors including MMR deficient endometrial cancer (Le et al, N. Engl. J. Med. 2015, 372:2509-2520). It is approved in the US for treatment of MSI-H or MMR deficient endometrial cancer that has progressed on prior therapy. The ORR for EC was 36% and duration of response ranged from 4-17 months.

However, a majority of EC is comprised of MSS tumors. There is an unmet need for more effective treatment of MSS endometrial cancer that has progressed following initial platinum-based chemotherapy. EC cells overexpress PD-1 and PD-L1 in 25-75% of cases, highest among all gynaecological cancers (Herzog et al, Gynecol. Oncol. 2015, 137:204-205). Clinical activity of monotherapy with anti PD-(L)-1 antibodies for MSS tumors without abnormalities in DNA repair is modest and no benefit on survival has been established (Ott et al, J. Immunother. Cancer, 2017, 5:16; Marcus et al, Clin. Cancer Res. 2019, 25:3753-3758; and Fleming et al, J. Clin. Onc. 2017, 35(15 suppl):Abstract 5585.

Combination therapies with anti PD-1 antibodies may be more effective. Recently, pembrolizumab in combination with lenvatinib has showed additional benefit in the MSI-H and MSS tumors following progression on prior systemic therapies with an overall response rate at week 24 in MSI-H tumors of 63.6% and 36.2% in participants with MSS tumors (Makker et al, J. Clin. Oncol. 2020; DOI: 10.1200/JCO.19.02627). Grade 3 or 4 adverse events were reported in 66.9% of participants and 21% discontinued treatement secondary to adverse events. More combination regimens need to be evaluated in this population to improve safety and efficacy of currently available therapies.

Further, endometrial cancer has been shown to have much higher amounts of indoleamine-2,3-dioxygenase (IDO) in inflamed tissue as compared to tryptophan-2,3-dioxygenase (TDO). IDO and TDO are the two major enzymes that regulate the first and rate-limiting step of the kynurenine pathway. As described above, local depletion of tryptophan and accumulation of proapoptotic kynurenines can greatly affect T-cell proliferation and survival. Therefore, cancers that express much higher amounts of IDO as compared to TDO may respond better to treatment with an IDO inhibitor and a PD-1 antibody, such as ANTIBODY X. Current translational data set shows that endometrial cancer expresses 40 times higher levels of IDO compared to TDO and 60% IDOhi/TDOlow, making it more susceptible to treatment with an IDO inhibitor such as epacadostat. Other cancers with high ratio of IDO:TDO include cervical cancer (IDO:TDO 79:1 and 60% IDOhi/TDOlow), renal cancer (or kidney renal clear cell carcinoma (KIRC) (IDO:TDO 45:1 and 60% IDOhi/TDOlow), lung cancer, including lung adenocarcinoma (IDO:TDO 7.5:1 and >25% IDOhi/TDOlow),

and head and neck cancer (head and neck squamous cell carcinoma) (IDO:TDO 8:1 and 20% IDOhi/TDOlow). As Example 1 shows that higher doses of epacadostat (up to 600 mg) results in sustained (up to 4 months) decreases in plasma kynurenine levels in most patients, these cancers should be more responsive to treatment with epacadostat than cancers with low levels of IDO compared with TPO.

### Inclusion Criteria

Participants are eligible to be included in the study only if all of the following criteria apply:
Ability to comprehend and willingness to sign a written ICF for the study.
Women 18 years of age or older (or as applicable per local country requirements).
Histologically confirmed diagnosis of advanced or metastatic endometrial cancer (other than carcinosarcoma and sarcoma of the uterus).
Radiologic evidence of disease progression after treatment with no more than 1 platinum-containing regimen for advanced or metastatic disease.
   One neoadjuvant/adjuvant chemotherapy in an early disease stage is allowable. Participants may receive up to 2 regimens of platinum-based chemotherapy in total, as long as one is given in the neoadjuvant or adjuvant treatment setting. Prior hormonal therapy is allowable in any disease setting.
Willing to provide tumor tissue sample (fresh or archived). Tumor tissue will be centrally tested for MSS and PD-L1 status.
   Tumors must be PD-L1 positive and MSS for enrollment on study as defined by central testing results.
Must have at least 1 measurable tumor lesion per RECIST v1.1.
ECOG performance status 0 or 1.
Willingness to avoid pregnancy based on the criteria below.
   Women of childbearing potential must have a negative serum pregnancy test at screening and must agree to take appropriate precautions to avoid pregnancy (with at least 99% certainty) from screening through 6 months after the last dose of study treatment. Permitted methods that are at least 99% effective in preventing pregnancy should be communicated to the participants and their understanding confirmed.
Women of nonchildbearing potential (i.e., surgically sterile with a hysterectomy and/or bilateral oophorectomy OR≥12 months of amenorrhea and at least 50 years of age) are eligible.

### Study Treatment Information

Table 6 describes the study treatment information. At visits where epacadostat is administered in the clinic, it should be administered just before the start of the ANTIBODY X infusion. Dose modification of ANTIBODY X and epacadostat are not permitted. If a dose interruption is necessary for management of drug-related TEAEs, ANTIBODY X will be reinitiated at 500 mg Q4W.

TABLE 6
Study Treatment Information

| | | |
|---|---|---|
| Study treatment name: | ANTIBODY X | Epacadostat |
| Mechanism of action: | PD-1 inhibitor | IDO1 inhibitor |
| Dosage formulation: | Liquid formulation | 300 mg tablets |
| Unit dose strength(s)/ | 500 mg Q4W | 600 mg BID |
| dosage level(s): | | |
| Administration | IV over 30 (+15) | 2 tablets twice |
| instructions: | minutes using a filter | daily without |
| | | regard to food |

### Example 3. Phase 2/3 Study of Retifanlimab Plus Epacadostat Versus Retifanlimab Plus Placebo in Participants with High Risk BCG-Unresponsive Non-Muscle Invasive Bladder Cancer

### General Study Design

This is a multicenter, randomized, double-blind, placebo-controlled, Phase 2/3 study of ANTIBODY X (i.e., retifanlimab) and epacadostat in participants with BCG-unresponsive, high-risk, non-muscle-invasive bladder cancer (NMIBC) with carcinoma in situ (CIS) with or without papillary tumors who are ineligible for or have elected not to undergo cystectomy conducted in conformance with Good Clinical Practices. Participants will be stratified by PD-L1 status (PD-L1 positive vs. PD-L1 negative) and by papillary disease status (papillary vs non-papillary disease present at baseline). The study consists of 2 treatment groups:

Group A: retifanlimab 500 mg Q4W plus placebo BID

Group B: retifanlimab 500 mg Q4W plus epacadostat 600 mg BID

This study will consist of 2 Phases. Phase 2 will begin with a 2:1 randomization for participants to receive retifanlimab and placebo or retifanlimab and epacadostat, respectively. After 150 participants have been enrolled there will be a pause in enrollment for participants to be monitored for response to treatment for up to 6 months. If the analysis at the end of Phase 2 meets desired criteria, the study will open Phase 3 enrollment with a 1:2 randomization for an additional 150 participants to receive retifanlimab and placebo or retifanlimab and epacadostat respectively.

After discontinuation of study treatment, the treatment portion of the study will end, and the participant will enter follow-up. Follow-up consists of 2 parts, safety follow-up, disease status follow-up. Participants are followed for safety for 90 days after the last dose of study treatment or until they begin a new anticancer therapy, whichever occurs first. Participants who discontinue study treatment for a reason other than disease progression will move into the disease status follow-up period and should continue to be assessed Q12W by efficacy assessments to monitor disease status until the start of a new anticancer therapy, disease progression, death, the end of the study, or the participant is lost to follow-up.

### Inclusion Criteria

Participants are eligible to be included in the study only if all of the following criteria apply:
1. Ability to comprehend and willingness to sign a written ICF for the study.
2. Men and women 18 years of age or older (or as applicable per local country requirements).
3. Pathologically confirmed high risk NMIBC defined as carcinoma-in-situ (CIS) with or without papillary tumors (High Grade Ta or T1),
   Predominant histologic component (>50%) must be urothelial (transitional cell) carcinoma
4. Demonstrated BCG-unresponsive (per February 2018 FDA guidance),
   BCG-unresponsive high-risk NMIBC is defined as: Persistent or recurrent CIS alone or with recurrent Ta/T1 (noninvasive papillary disease/tumor invades the subepithelial connective tissue) disease within 12 months of completion of adequate BCG therapy. Adequate BCG therapy is defined as a minimum of 5 of 6 doses of an induction course (adequate induction) plus 2 of 3 doses of a maintenance course, or 2 of 6 doses of a second induction course.
5. Underwent ≥2 cystoscopic procedures, with the most recent ≤8 weeks before study start confirming high risk NMIBC as defined in inclusion criteria #4 is present, including complete TURBT.
6. Fully resected papillary disease at study entry; residual CIS acceptable.
7. Willing to provide tumor tissue sample (archival or fresh biopsy containing CIS). Archival tissue must be available and sufficient for biomarker analyses. Samples should be within 6 months of screening and include tissue representative from each part of the bladder that is suspicious for CIS disease.
8. Ineligible for or elected not to undergo radical cystectomy.
9. ECOG performance status 0 to 1.
10. Willingness to avoid pregnancy based on the criteria below.
   Male participants with childbearing potential must agree to take appropriate precautions to avoid fathering children (with at least 99% certainty) from screening through 90 days after the last dose of study treatment and must refrain from donating sperm during this period. Permitted methods that are at least 99% effective in preventing pregnancy should be communicated to the participants and their understanding confirmed.
   Women of childbearing potential must have a negative serum pregnancy test at screening and must agree to take appropriate precautions to avoid pregnancy (with at least 99% certainty) from screening through 6 months after the last dose of study treatment. Permitted methods that are at least 99% effective in preventing pregnancy should be communicated to the participants and their understanding confirmed.
   Women of nonchildbearing potential (i.e., surgically sterile with a hysterectomy and/or bilateral oophorectomy OR≥12 months of amenorrhea and at least 50 years of age) are eligible.

### Study Treatment Information

Table 7 describes presents the study treatment information for retifanlimab and epacadostat, respectively. At visits where epacadostat is administered in the clinic, it should be administered just before the start of the retifanlimab infusion. Dose modification of retifanlimab and epacadostat are not permitted.

TABLE 7
Study Treatment Information

| | | |
|---|---|---|
| Study treatment name: | Retifanlimab | Epacadostat |
| Mechanism of action: | PD-1 inhibitor | IDO1 inhibitor |
| Dosage formulation: | Liquid formulation | 300 mg tablets |
| Unit dose strength(s)/ | 500 mg Q4W | 600 mg BID |
| dosage level(s): | | |
| Administration | IV over 30 (+15) | 2 tablets taken |
| instructions: | minutes using a filter | orally BID without |
| | | regard to food |

Unless otherwise defined, all technical and scientific terms used herein have the same meaning as commonly understood by one of ordinary skill in the art to which this invention belongs. Although methods and materials similar or equivalent to those described herein can be used in the practice or testing of the present disclosure, the exemplary methods and materials are described below. All publications, patent applications, patents, and other references mentioned herein are incorporated by reference in their entirety. In case of conflict, the present disclosure, including definitions, will control. The materials, methods, and examples are illustrative only and not intended to be limiting.

What is claimed is:

1. A method of treating cancer in a patient, comprising administering to said patient:
(i) epacadostat, or a pharmaceutically acceptable salt thereof, at a dose from about 400 mg to about 700 mg on a free base basis BID; and
(ii) an antibody, or an antigen-binding fragment thereof, that binds to human PD-1, wherein the antibody comprises (ii-1) a variable heavy (VH) domain comprising VH complementarity determining region (CDR)1, VH CDR2, and VH CDR3; and (ii-2) a variable light (VL) domain comprising VL CDR1, VL CDR2, and VL CDR3; wherein:
(a) the VH CDR1 comprises the amino acid sequence SYWMN (SEQ ID NO:6);
(b) the VH CDR2 comprises the amino acid sequence VIHPSDSETWLDQKFKD (SEQ ID NO:7);
(c) the VH CDR3 comprises the amino acid sequence EHYGTSPFAY (SEQ ID NO:8);
(d) the VL CDR1 comprises the amino acid sequence RASESVDNYGMSFMNW (SEQ ID NO:9);
(e) the VL CDR2 comprises the amino acid sequence AASNQGS (SEQ ID NO:10); and
(f) the VL CDR3 comprises the amino acid sequence QQSKEVPYT (SEQ ID NO:11).

2. The method of claim 1, wherein the epacadostat is administered as the free base.

3. The method of claim 2, wherein the epacadostat is administered at a dose of about 400 mg BID.

4. The method of claim 2, wherein the epacadostat is administered at a dose of about 425 mg BID.

5. The method of claim 2, wherein the epacadostat is administered at a dose of about 450 mg BID.

6. The method of claim 2, wherein the epacadostat is administered at a dose of about 475 mg BID.

7. The method of claim 2, wherein the epacadostat is administered at a dose of about 500 mg BID.

8. The method of claim 2, wherein the epacadostat is administered at a dose of about 525 mg BID.

9. The method of claim 2, wherein the epacadostat is administered at a dose of about 550 mg BID.

10. The method of claim 2, wherein the epacadostat is administered at a dose of about 575 mg BID.

11. The method of claim 2, wherein the epacadostat is administered at a dose of about 600 mg BID.

12. The method of claim 1, wherein the antibody is administered at a dose of about 500 mg.

13. The method of claim 1, wherein the antibody is administered at a fixed dose of about 500 mg once every four weeks.

14. The method of claim 1, wherein the antibody is administered at a fixed dose of about 375 mg once every 3 weeks.

15. The method of claim 1, wherein the antibody is administered via intravenous administration.

16. The method of claim 1, wherein the VH domain comprises the amino acid sequence set forth in SEQ ID NO:4.

17. The method of claim 1, wherein the antibody comprises a heavy chain and wherein the heavy chain comprises the amino acid sequence set forth in SEQ ID NO:2.

18. The method of claim 1, wherein the VL domain comprises the amino acid sequence set forth in SEQ ID NO:5.

19. The method of claim 1, wherein the antibody comprises a light chain and wherein the light chain comprises the amino acid sequence set forth in SEQ ID NO:3.

20. The method of claim 1, wherein the VH domain comprises the amino acid sequence set forth in SEQ ID NO:4 and the VL domain comprises the amino acid sequence set forth in SEQ ID NO:5.

21. The method of claim 1, wherein the antibody comprises a heavy chain and a light chain, and wherein the heavy chain comprises the amino acid sequence set forth in SEQ ID NO:2 and the light chain comprises the amino acid sequence set forth in SEQ ID NO:3.

22. The method of claim 1, wherein the antibody comprises an Fc Region of the IgG4 isotype and an IgG4 Hinge Domain that comprises a stabilizing mutation.

23. The method of claim 1, wherein the antibody is a humanized antibody.

24. The method of claim 1, wherein the cancer is a solid tumor.

25. The method of claim 1, wherein the cancer is skin cancer, lung cancer, lymphoma, sarcoma, bladder cancer, cancer of the ureter, urethra, and urachus, gastric cancer, cervical cancer, liver cancer, breast cancer, renal cancer, head and neck cancer, squamous cell carcinoma, colorectal cancer, endometrial cancer, anal cancer, and a tumor with microsatellite instability-high (MSI-H), mismatch repair deficient (dMMR) or DNA polymerase ε exonuclease domain mutation positive disease.

26. The method of claim 1, wherein the cancer is cholangiocarcinoma, melanoma, non-small cell lung cancer, small cell lung cancer, Hodgkin's lymphoma, urothelial carcinomagastric cancer, hepatocellular carcinoma, Merkel cell carcinoma, triple-negative breast cancer, renal cell carcinoma, squamous cell carcinoma of the head and neck, and colorectal cancer.

27. The method of claim 1, wherein the cancer is squamous cell carcinoma of the anal canal.

28. The method of claim 1, wherein the cancer is Merkel cell carcinoma.

29. The method of claim 1, wherein the cancer is endometrial cancer.

30. The method of claim 1, wherein the cancer is cervical cancer.

31. The method of claim 1, wherein the cancer is renal cancer.

32. The method of claim 31, wherein the cancer is kidney renal clear cell carcinoma.

33. The method of claim 1, wherein the cancer is lung cancer.

34. The method of claim 33, wherein the cancer is adenocarcinoma of the lung.

35. The method of claim 33, wherein the cancer is squamous cell carcinoma of the lung.

36. The method of claim 33, wherein the cancer is non-small cell lung cancer.

37. The method of claim 1, wherein the cancer is head and neck cancer.

38. The method of claim 37, wherein the cancer is head and neck squamous cell carcinoma.

39. The method of claim 1, wherein the cancer is bladder cancer.

40. The method of claim 39, wherein the bladder cancer is high risk BCG-unresponsive non-muscle invasive bladder cancer.

41. The method of claim 1, wherein the cancer is microsatellite-stable (MSS).

42. The method of claim 1, wherein the cancer is PD-L1 positive.

43. The method of claim 1, wherein the cancer is microsatellite-stable (MSS) and PD-L1 positive.
